(19) United States
(12) Patent Application Publication
(10) Pub. No.: US 2023/0391886 A1
(43) Pub. Date: Dec. 7, 2023

(54) COMPOSITIONS AND METHODS FOR MUC18 TARGETING

(71) Applicant: BOARD OF REGENTS, THE UNIVERSITY OF TEXAS SYSTEM, Austin, TX (US)

(72) Inventors: Jeffrey LEE, Houston, TX (US); Mason LU, Fremont, CA (US); Runhua FENG, Shanghai (CN); Yuling WANG, Houston, TX (US); Vijaya RAMACHANDRAN, Pearland, TX (US)

(73) Assignee: BOARD OF REGENTS, THE UNIVERSITY OF TEXAS SYSTEM, Austin, TX (US)

(21) Appl. No.: 18/249,241

(22) PCT Filed: Oct. 15, 2021
(86) PCT No.: PCT/US2021/055320

§ 371 Date: Apr. 14, 2023

Related U.S. Application Data
(60) Provisional application No. 63/093,024, filed on Oct. 16, 2020.

(51) Int. Cl. C07K 16/30; A61P 35/00

(52) U.S. Cl. CPC C07K 16/3092; A61P 35/00; C07K 2317/92; A61K 2039/505; C07K 2317/24; C07K 2317/34; C07K 2317/20

(57) ABSTRACT

Aspects of the disclosure relate to MUC18-binding proteins. Embodiments include methods for treating one or more conditions, for example cancer, using a MUC18-binding protein. In some embodiments, the disclosed methods and compositions involve one or more antibodies that are capable of binding MUC18.

This application claims benefit of priority of U.S. Provisional Application No. 63/093,024, filed Oct. 16, 2020, which is hereby incorporated by reference in its entirety.

This invention was made with government support under grant numbers CA093459 and CA016672 awarded by the National Institutes of Health. The government has certain rights in the invention.

### SEQUENCE LISTING

The instant application contains a Sequence Listing which has been submitted in ASCII format and is hereby incorporated by reference in its entirety. Said ASCII copy, created on Oct. 5, 2021, is named MDAC.P1254WO_Sequence_Listing.txt and is 12,605 bytes in size.

### BACKGROUND

### Field of the Invention

This invention relates generally to the fields of molecular biology, immunology, immunotherapy, and medicine.

### Background

Melanoma has the fastest growing incidence among all cancers in the United States. Melanoma accounts for more than 75% of annual skin cancer deaths and is the leading cause of cancer-related death in young women. Promising therapies available for patients with aggressive melanoma include those that target specific common tumor mutations (e.g., BRAF and MEK inhibitors) or enhance anti-tumor immunity (e.g., anti-CTLA4 and anti-PD1/anti-PDL1). However, even these therapies are usually only partially and/or temporarily effective, are effective in a minority of patients, and can have significant associated side effects. Thus, there is a need in the art for methods and compositions for targeting novel mechanisms for treating cancer with greater efficacy in a majority of patients with fewer associated side effects.

### SUMMARY

Described herein, in some aspects, are methods and compositions for effectively treating subjects with cancer (e.g., melanoma) using polypeptides (e.g., antibodies) targeting MUC18. In certain aspects, the MUC18-binding proteins, for example MUC18-neutralizing antibodies, described herein are unique in that they can specifically bind a glycosylated, conformational, tumor-specific epitope involving sialic acid on MUC18 expressed on the cell surface of melanoma cells, resulting in a reduction of off-target effects. Thus, the methods and compositions described herein can eliminate or reduce side effects associated with other available cancer therapies. Additionally, the tumor-specific MUC18 neutralizing antibodies described herein can functionally inhibit in vitro melanoma cell growth, in vivo xenograft melanoma tumor growth, and migration/invasion and metastasis both in vitro and in vivo, thereby providing efficacious cancer treatment.

Embodiments of the present disclosure include, inter alia, antigen-binding proteins (e.g., antibodies, antibody-like molecules, or fragments thereof) comprising a heavy chain variable region (VH) having 85%, 86%, 87%, 88%, 89%, 90%, 92%, 93%, 94%, 95%, 96%, 97%, 98%, 99%, or 100% sequence identity, or any range derivable therein, with SEQ ID NO:20 and a light chain variable region (VL) having 85%, 86%, 87%, 88%, 89%, 90%, 92%, 93%, 94%, 95%, 96%, 97%, 98%, 99%, or 100% sequence identity, or any range derivable therein, with SEQ ID NO:21. In some embodiments, the VH has at least 85% identity with SEQ ID NO:20 and the VL has at least 85% identity with SEQ ID NO:21. In some embodiments, the VH has at least 95% identity with SEQ ID NO:20 and the VL has at least 95% identity with SEQ ID NO:21. In some embodiments, the VH comprises SEQ ID NO:20 and the VL comprises SEQ ID NO:21. In some embodiments, the disclosed antigen-binding proteins, antibodies, antibody-like molecules, and fragments thereof are melanoma cellular adhesion molecule (MUC18)-binding proteins (i.e., are capable of binding to MUC18). In some embodiments, disclosed herein are monoclonal anti-MUC18 antibodies. In some embodiments, disclosed herein are humanized anti-MUC18 antibodies. Antigen-binding proteins described herein may be used in treating one or more conditions associated with expression or activity of a MUC18 protein such as, for example, cancer. In some embodiments, MUC18-binding proteins are be used in treating one or more MUC18-associated conditions with reduced risk of toxicity and improved efficacy as compared to previously disclosed MUC18-binding proteins.

Embodiments include compositions comprising one or more antigen-binding proteins (e.g., MUC18-binding proteins). Embodiments include an antigen-binding protein comprising one or more regions (e.g., heavy chain variable region, light chain variable region, etc.). Embodiments include monoclonal antibodies, humanized antibodies, or antibody-like molecules. Embodiments also include nucleic acid molecules encoding for one or more antigen-binding proteins or portions thereof. Embodiments include recombinant, transformed, or modified cells, vectors, and/or expression cassettes comprising such nucleic acid molecules. In some embodiments, the compositions contemplated herein can comprise 1, 2, 3, 4, 5, or more of the following components: an antigen-binding protein, a nucleic acid, a vector, a cell, a polypeptide, an oligonucleotide, a light chain variable region, a heavy chain variable region, a light chain constant region, and a heavy chain constant region. Any one or more of these components may be excluded from the disclosed compositions.

Embodiments also include methods of generating an antigen-binding protein, methods of producing an antigen-binding protein, methods of expressing an antigen-binding protein, methods of antigen-binding proteins, methods of detecting MUC18, methods of treating one or more conditions, methods of purifying MUC18, methods of treating cancer, and methods of eliminating one or more cells expressing MUC18. The steps and embodiments discussed in this disclosure are contemplated as part of any of these methods. In some embodiments, the methods contemplated herein can comprise or exclude 1, 2, 3, 4, 5, or more of the following steps: providing an antigen-binding protein, providing a nucleic acid to a cell, subjecting a cell to conditions sufficient to express a nucleic acid, providing an additional therapeutic, covalently attaching a therapeutic to an antigen-binding protein, non-covalently attaching a therapeutic to an antigen-binding protein, expressing a vector in a cell, and providing a pharmaceutical composition to a subject. Any one or more of these steps may be excluded from the disclosed methods.

In some aspects, the disclosure relates to a polypeptide that specifically binds MUC18 comprising (a) a VH comprising: (i) a first complementarity determining region (CDR-H1) amino acid sequence having 85%, 86%, 87%, 88%, 89%, 90%, 92%, 93%, 94%, 95%, 96%, 97%, 98%, 99%, or 100% sequence identity, or any range derivable therein, with SEQ ID NO:1, SEQ ID NO:2, or SEQ ID NO:3; (ii) a second CDR (CDR-H2) amino acid sequence having 85%, 86%, 87%, 88%, 89%, 90%, 92%, 93%, 94%, 95%, 96%, 97%, 98%, 99%, or 100% sequence identity, or any range derivable therein, with SEQ ID NO:4, SEQ ID NO:5, or SEQ ID NO:6; and a third CDR (CDR-H3) amino acid sequence having 85%, 86%, 87%, 88%, 89%, 90%, 92%, 93%, 94%, 95%, 96%, 97%, 98%, 99%, or 100% sequence identity, or any range derivable therein, with SEQ ID NO:7, SEQ ID NO:8, SEQ ID NO:9, or SEQ ID NO:10; and (b) a VL comprising: (i) a first CDR (CDR-L1) amino acid sequence having 85%, 86%, 87%, 88%, 89%, 90%, 92%, 93%, 94%, 95%, 96%, 97%, 98%, 99%, or 100% sequence identity, or any range derivable therein, with SEQ ID NO:11, SEQ ID NO:12, or SEQ ID NO:13; (ii) a second CDR (CDR-L2) amino acid sequence having at 85%, 86%, 87%, 88%, 89%, 90%, 92%, 93%, 94%, 95%, 96%, 97%, 98%, 99%, or 100% sequence identity, or any range derivable therein, with SEQ ID NO:14, SEQ ID NO:15, or SEQ ID NO:16; and a third CDR (CDR-L3) amino acid sequence having 85%, 86%, 87%, 88%, 89%, 90%, 92%, 93%, 94%, 95%, 96%, 97%, 98%, 99%, or 100% sequence identity, or any range derivable therein, with SEQ ID NO:17, SEQ ID NO:18, or SEQ ID NO:19.

In some aspects, the disclosure relates to a polypeptide that specifically binds MUC18 comprising (a) a VH comprising: (i) a CDR-H1 amino acid sequence having at least 85% identity with SEQ ID NO:1, SEQ ID NO:2, or SEQ ID NO:3; (ii) a CDR-H2 amino acid sequence having at least 85% identity with SEQ ID NO:4, SEQ ID NO:5, or SEQ ID NO:6; and a CDR-H3 amino acid sequence having at least 85% identity with SEQ ID NO:7, SEQ ID NO:8, SEQ ID NO:9, or SEQ ID NO:10; and (b) a VL comprising: (i) a CDR-L1 amino acid sequence having at least 85% identity with SEQ ID NO:11, SEQ ID NO:12, or SEQ ID NO:13; (ii) a CDR-L2 amino acid sequence having at least 85% identity with SEQ ID NO:14, SEQ ID NO:15, or SEQ ID NO:16; and a CDR-L3 amino acid sequence having at least 85% identity with SEQ ID NO:17, SEQ ID NO:18, or SEQ ID NO:19.

In some aspects, the disclosure relates to a polypeptide that specifically binds MUC18 comprising (a) a VH comprising: (i) a CDR-H1 amino acid sequence having at least 95% identity with SEQ ID NO:1, SEQ ID NO:2, or SEQ ID NO:3; (ii) a CDR-H2 amino acid sequence having at least 95% identity with SEQ ID NO:4, SEQ ID NO:5, or SEQ ID NO:6; and a CDR-H3 amino acid sequence having at least 95% identity with SEQ ID NO:7, SEQ ID NO:8, SEQ ID NO:9, or SEQ ID NO:10; and (b) a VL comprising: (i) a CDR-L1 amino acid sequence having at least 95% identity with SEQ ID NO:11, SEQ ID NO:12, or SEQ ID NO:13; (ii) a CDR-L2 amino acid sequence having at least 95% identity with SEQ ID NO:14, SEQ ID NO:15, or SEQ ID NO:16; and a CDR-L3 amino acid sequence having at least 95% identity with SEQ ID NO:17, SEQ ID NO:18, or SEQ ID NO:19.

In some aspects, the disclosure relates to a polypeptide that specifically binds MUC18 comprising (a) a VH comprising: (i) a CDR-H1 amino acid sequence comprising SEQ ID NO:1, SEQ ID NO:2, or SEQ ID NO:3; (ii) a CDR-H2 amino acid sequence comprising SEQ ID NO:4, SEQ ID NO:5, or SEQ ID NO:6; and a CDR-H3 amino acid sequence comprising SEQ ID NO:7, SEQ ID NO:8, SEQ ID NO:9, or SEQ ID NO:10; and (b) a VL comprising: (i) a CDR-L1 amino acid sequence comprising SEQ ID NO:11, SEQ ID NO:12, or SEQ ID NO:13; (ii) a CDR-L2 amino acid sequence comprising SEQ ID NO:14, SEQ ID NO:15, or SEQ ID NO:16; and a CDR-L3 amino acid sequence comprising SEQ ID NO:17, SEQ ID NO:18, or SEQ ID NO:19.

In some embodiments, the MUC18-binding protein has an affinity for MUC18. In some embodiments, the MUC18-binding protein has an affinity for MUC18 of between 0.001 and 1000 nM. In some embodiments, the MUC18-binding protein has an affinity for MUC18 of between 0.01 and 100 nM. In some embodiments, the MUC18-binding protein has an association constant for a MUC18 protein of between 0.1 and 20 nM. In some embodiments, the MUC18-binding protein has an association constant for a MUC18 protein of between 1 and 10 nM. In some embodiments, the MUC18-binding protein has an association constant for a MUC18 protein of between 1 and 3 nM. In some embodiments, the MUC18-binding protein has an association constant for a MUC18 protein of at most, at least, or about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 20.5, 21, 21.5, 22, 22.5, 23, 23.5, 24, 24.5, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, or 1000 nM, or any range derivable therein.

In some embodiments, the MUC18-binding protein specifically binds to an extracellular domain of MUC18. In some embodiments, the MUC18-binding protein preferentially binds to a glycosylated MUC18 compared to a non-glycosylated MUC18.

In some embodiments, the MUC18-binding protein is an antibody, an antibody-like molecule, or an antigen-binding fragment thereof. In some embodiments, the MUC18-binding protein is an antibody, a nanobody, a minibody, an scFv fragment, or an Fab fragment. In some embodiments, the MUC18-binding protein is a human antibody, humanized antibody, recombinant antibody, chimeric antibody, an antibody derivative, a veneered antibody, a diabody, a monoclonal antibody, or a polyclonal antibody. In some embodiments, the MUC18-binding protein is a monoclonal antibody. In some embodiments, the MUC18-binding protein is a murine antibody. In some embodiments, the MUC18-binding protein is JM1-24-2. In some embodiments, the MUC18-binding protein is a humanized antibody. In some embodiments, the MUC18-binding protein is a human antibody.

Some aspects are directed to a nucleic acid encoding for a polypeptide that specifically binds MUC18. In some embodiments, the nucleic acid comprises a nucleotide sequence having 85%, 86%, 87%, 88%, 89%, 90%, 92%, 93%, 94%, 95%, 96%, 97%, 98%, 99%, or 100% sequence identity, or any range derivable therein, with SEQ ID NO:22. In some embodiments, the nucleic acid comprises a nucleotide sequence having at least 85% identity to SEQ ID NO:22. In some embodiments, the nucleic acid comprises a nucleotide sequence having at least 95% identity to SEQ ID NO:22. In some embodiments, the nucleic acid comprises SEQ ID NO:22. In some embodiments, the nucleic acid comprises a nucleotide sequence having 85%, 86%, 87%, 88%, 89%, 90%, 92%, 93%, 94%, 95%, 96%, 97%, 98%, 99%, or 100% sequence identity, or any range derivable therein, with SEQ ID NO:23. In some embodiments, the nucleic acid comprises a nucleotide sequence having at least 85% identity to SEQ ID NO:23. In some embodiments, the nucleic acid comprises a nucleotide sequence having at least 95% identity to SEQ ID NO:23. In some embodiments, the nucleic acid comprises SEQ ID NO:23. Also disclosed is a vector comprising the nucleic acid.

Certain aspects are directed to a cell comprising a polypeptide that specifically binds MUC18 (the MUC18-binding protein), a nucleic acid, and/or a vector of the present disclosure. In some embodiments, the cell comprises the MUC18-binding protein and is capable of secreting the MUC18-binding protein outside the cell. In some embodiments, the cell comprises the MUC18-binding protein and the MUC18-binding protein is attached to a surface of the cell. In some embodiments, the MUC18-binding protein is a chimeric antigen receptor. In some embodiments, the MUC18-binding protein is a T cell receptor. In some embodiments, the cell is an immune cell. In some embodiments, the cell is a B cell. In some embodiments, the cell is a T cell.

Certain aspects are directed to a composition comprising a MUC18-binding protein, such as a MUC18-binding protein described herein, a nucleic acid encoding for the MUC18-binding protein, a vector comprising the nucleic acid, or a cell comprising the MUC18-binding protein, the nucleic acid, or the vector; and a pharmaceutically acceptable excipient. In some embodiments, the composition further comprises an additional therapeutic. In some embodiments, the additional therapeutic is covalently attached to the MUC18-binding protein. In some embodiments, the additional therapeutic is non-covalently attached to the MUC18-binding protein. In some embodiments, the additional therapeutic is a biotherapeutic, a chemotherapeutic drug, an immunotherapeutic, a toxin, an antisense oligonucleotide, a small inhibitory RNA (siRNA), an enzyme, a protein (e.g., a toxin, an enzyme, etc.), a viral vector, or a nanodrug.

Other embodiments are directed to a use of a composition comprising a polypeptide that specifically binds MUC18 which in some embodiments comprises an additional therapeutic agent, in the manufacture of a medicament for the treatment or prevention of cancer. Certain embodiments are directed to use of the composition comprising the MUC18-binding protein in the manufacture of a medicament for the treatment or prevention of cancer.

A further embodiment is directed to a polypeptide that specifically binds MUC18 prepared by a method described herein. Some aspects are directed to a method for generating a MUC18-binding protein, such as a MUC18-binding protein described herein, by culturing a cell described herein under conditions sufficient to express a nucleic acid disclosed herein in the cell. Some aspects are directed to a method for generating a MUC18-binding protein, such as a MUC18-binding protein described herein, comprising (a) providing to a cell a nucleic acid encoding for the MUC18-binding protein; and (b) subjecting the cell to conditions sufficient to express the nucleic acid in the cell.

In some embodiments, provided herein is a method for treating cancer in a subject comprising providing to the subject a therapeutically effective amount of a MUC18-binding protein, such as a MUC18-binding protein described herein, a nucleic acid encoding for the MUC18-binding protein, a vector comprising the nucleic acid, or a cell comprising the MUC18-binding protein, the nucleic acid, or the vector. In some embodiments, the method further comprises providing to the subject one or more additional therapeutics. In some embodiments, the one or more additional therapeutics are covalently attached to the MUC18-binding protein. In some embodiments, the one or more additional therapeutics are non-covalently attached to the MUC18-binding protein. In some embodiments, the one or more additional therapeutics comprise a radiotherapeutic, a biotherapeutic, an immunotherapeutic, a chemotherapeutic, a toxin, an antisense oligonucleotide, a small inhibitory RNA (siRNA), a protein (e.g., a toxin or an enzyme), a viral vector, or a nanodrug.

Some embodiments are directed to an anti-MUC18 antibody or antigen-binding fragment thereof comprising: (a) a VH comprising (i) a CDR-H1 amino acid sequence comprising SEQ ID NO:1; (ii) a CDR-H2 amino acid sequence comprising SEQ ID NO:4; and (iii) a CDR-H3 amino acid sequence comprising SEQ ID NO:7; and (b) a VL comprising: (i) a CDR-L1 amino acid sequence comprising SEQ ID NO:11; (ii) a CDR-L2 amino acid sequence comprising SEQ ID NO:14; and (iii) a CDR-L3 amino acid sequence comprising SEQ ID NO:17. In some embodiments, the VH comprises SEQ ID NO: 20. In some embodiments, the VL comprises SEQ ID NO: 21. In some embodiments, the anti-MUC18 antibody or antigen-binding fragment has an association constant for a MUC18 protein of between 1 and 3 nM.

Throughout this application, the term “about” is used to indicate that a value includes the inherent variation of error for the device, the method being employed to determine the value, or the variation that exists among the study subjects.

The use of the word “a,” “an,” and “the” when used in conjunction with the term “comprising” may mean “one,” but it is also consistent with the meaning of “one or more,” “at least one,” and “one or more than one.” Thus, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

As used herein “another” may mean at least a second or more.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Thus, as used herein the term “comprising” or “comprises” is used in reference to compositions, methods, and respective component(s) thereof, that are essential to the invention, yet open to the inclusion of unspecified elements, whether essential or not.

The compositions and methods for their use can “comprise,” “consist essentially of,” or “consist of” any of the ingredients or steps disclosed throughout the specification. As used herein the term “consisting essentially of” refers to those elements required for a given embodiment. The term permits the presence of additional elements that do not materially affect the basic and novel or functional characteristic(s) of that embodiment of the invention. With respect to pharmaceutical compositions, the term “consisting essentially of” includes the active ingredients recited, excludes any other active ingredients, but does not exclude any pharmaceutical excipients or other components that are not therapeutically active. The term “consisting of” refers to compositions, methods, and respective components thereof as described herein, which are exclusive of any element not recited in that description of the embodiment.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is contemplated that any method or composition described herein can be implemented with respect to any other method or composition described herein and that different embodiments may be combined.

Use of the one or more sequences or compositions may be employed based on any of the methods described herein. Other embodiments are discussed throughout this application. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. The embodiments in the Example section are understood to be embodiments that are applicable to all aspects of the technology described herein.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D show the screening and identification of an anti-MUC18 monoclonal antibody (mAb). Binding of JM1-24-3 to its target on melanoma cell surface was verified by FACS and ELISA. FIG. 1A. FACS analysis of JM1-24-3 binding on the surfaces of live melanoma cells showed that high metastatic cell lines A2058 and WM266-4 had significantly higher binding compared to low metastatic cell line A375. FIG. 1B. ELISA analysis of JM1-24-3 binding with the immobilized melanoma cell lysates showed similar binding pattern as of FACS. High metastatic cell line A2058 showed the maximum binding. MUC18 protein was defined as the target of JM1-24-3 by immunoprecipitation (IP) and mass spectrometry (MS) analyses. FIG. 1C. Immunoprecipitation of WM266-4 lysates with JM1-24-3 showed two bands—135 kD (high intensity) and 110 kD (faint band) by SDS-PAGE Coomassie blue staining. FIG. 1D. MS analysis showed that MUC18 glycoprotein had invariably the highest score among all the hits for both the bands.

FIGS. 2A-2E identify the carbohydrate moiety(ies) and define the conformational epitope of MUC18. JM1-24-3 interacts with the glycosylated MUC18 protein expressed on the cell surface of melanoma cells. Treatment of WM266-4 cells with tunicamycin significantly reduced JM1-24-3 binding with MUC18 as shown by FACS (p<0.01) FIG. 2A and showed reduced forms of MUC18 (110 and 90 kDa) by WB FIG. 2B. FLISA competition assay with WM266-4 cell membrane fraction showed that JM1-24-3 binding to MUC18 glycoprotein is significantly competed by lectin SNL (FIG. 2C). JM1-24-3 binds to the epitopes of MUC18 on melanoma cell surface. JM1-24-3 binding with WM266-4 is competitively reduced by MUC18 binding peptides, P1-BSA, P2-BSA and P3-BSA individually as shown by serial dilutions of Ab FIG. 2D or in combination FIG. 2E as shown by serial dilutions of peptides by FACS. The competitive interference achieved with combination of three peptides was greater than that achieved with the individual peptides.

FIGS. 3A-3D show that JM1-24-3 binds to MUC18 on melanoma cells and stimulates downstream signaling pathways in RPPA and computational models. FIG. 3A shows heat maps illustrating the differences in the expression of proteins in WM266-4 cell lysates with 1 hour/6 hour/no treatment with JM1-24-3 as analyzed by RPPA. FIG. 3B shows fold changes for up- or down-regulated proteins in RPPA as analyzed with the IPA software. WM266-4 cells treated with JM1-24-3/irrelevant mAb/PBS at different time points (30 minutes-24 hour) showed by WB that p-AKT (Ser473) and p-mTOR (Ser2448) had time-dependent reduction in phosphorylation until 6 hours, while both total AKT and mTOR remained unchanged and 3-actin served as loading control FIG. 3C. FIG. 3D provides structural models showing the conformation of the MUC18 epitope and relative binding of the heavy-chain, light-chain Fv peptides of JM1-24-3 to the flank and near side of the “bent” conformation compared to binding of the single-chain variable fragment (scFv) of JM1-24-3 to the top surface of MUC18 molecule when in its “extended” conformation.

FIGS. 4A-4C show that JM1-24-3 inhibits the proliferation, migration and invasion of melanoma cells. FIG. 4A. Treatment with JM1-24-3 (150 μg/mL) for seven days resulted in significant reduction in proliferation in A375 (52%), A2058 (76%) and WM266-4 (46%) cells as compared to irrelevant mAb treatment (p<0.02). Colorimetric assays on the migration of the WM266-4 cells treated with JM1-24-3 (150 μg/ml) showed significant reduction in migration assays (58%) FIG. 4B and in invasion assays (52%) FIG. 4C.

FIGS. 5A-5B show that JM1-24-3 inhibits melanoma tumor growth and reduces lung metastasis in xenograft athymic nude mice. FIG. 5A. Sub-cutaneous tumors developed with WM266-4 cells on athymic nude mice and treated with JM1-24-3 (n=11) or with irrelevant mAb (n=8) (6 mg/kg body wt/i.p.) twice a week; tumor volume was measured every 4 days until day 45. Treatment with JM1-24-3 showed significant reduction in tumor volume (46.9±11.8%; p<0.01). FIG. 5B. Pretreatment with JM1-24-3 (n=5) or irrelevant mAb (n=7) (6 mg/kg body wt/i.p/twice per week) done one day before tail vein injection of WM266-4 cells on athymic nude mice followed by treatment till 45 days. All mice were sacrificed at day 45 and their lungs were harvested and stained with H&E and the number of metastatic tumor colonies was counted. Treatment with JM1-24-3 showed significantly fewer colonies (p<0.05).

FIGS. 6A-6E show that the expression levels of MUC18 in cancers have clinical significance. FIG. 6A. The copy numbers of MUC18 mRNA were analyzed across a variety of cancers (red color) and normal tissues (black color) in TCGA cohort studies. The MUC18 gene expression was elevated in melanoma (SKCM) and renal cell carcinoma (KIRC). FIG. 6B. The MUC18 gene expression level in melanoma (SKCM) was five times as much as that in normal lung and prostate tissues. FIG. 6C. IHC of normal tissue microarray with JM1-24-3, showed positive staining on smooth muscle cells in small vessels of kidney, lung and skin, but not on vessels larger than small capillaries, while other normal tissues were negative. FIG. 6D. MUC18 IHC images with JM1-24-3 showing variable staining intensity from negative (upper left panel) to strong positive (lower right panel) on melanoma patient tissue slides. FIG. 6E. Staining intensity correlation of eight melanoma patients showed that metastatic melanoma patients had higher intensity staining of MUC18 with JM1-24-3 mAb. Also, all metastatic patients (5/5) showed stronger intensity.

FIG. 7 shows immunoprecipitation of melanoma cell lysates with irrelevant control abs and JM1-24-3 mAb. Lysates of A2058, WM266-4 and A375 (25 μg) were immunoprecipitated individually with 1 μg each of JM1-24-3 and other irrelevant Abs (1 & 2) followed by SDS-PAGE. One stronger band at 135 kD alone was observed on IP with JM1-24-3 mAb, while no band was observed with other irrelevant antibodies. PBMC served as lysate control and j-actin (42 kD) served as loading control.

FIGS. 8A-8F show the definition of MUC18 molecule and the carbohydrate group of glycoprotein in FLISA analyses. FIG. 8A. The lectin (DSA-FITC, LCA-FITC, WGA-FITC) binding to the lysate of A375 cell line, which were individually competed by JM1-24-3, each lectin plus JM1-24-3 and the buffer control to quantify MFI of MUC18 glycoprotein from the extraction of cell membrane proteins showed no competitive reduction in binding. FIG. 8B. JM1-24-3 binds to the epitopes of MUC18 on melanoma cell surface. Single-amino acid resolution map of the epitope recognized by JM1-24-3 on a peptide tiling array. The MFI and the binding peaks were visualized with anti-mouse IgG conjugated to Alexa Fluor® 647. FIG. 8C. Mapping of the peptide sequences recognized by JM1-24-3 to the MUC18 protein sequence. JM1-24-3 and commercial antibodies bound to the MUC18 molecule in different formats. FIG. 8D. Detection of MUC18 expression on the cell surface of melanoma cell line, WM266-4 with JM1-24-3.mouse anti-human MUC18 mAb, irrelevant mAb (negative control), goat anti-human MUC18 pAb and goat serum (IgG, negative control) by FACS analysis. FIG. 8E. Indirect ELISA showing binding to γHuMCAM by JM1-24-3, mouse anti-human MUC18 mAb and goat anti-human MUC18 pAb. FIG. 8F. Competition of binding between HRP-conjugated JM1-24-3 and γHuMCAM (left panel) or WM266-4 cell lysates by unconjugated JM1-24-3 (self-competition, right panel) and mouse anti-human MUC18 mAb, respectively, in ELISA assays. The irrelevant mAb and BSA/PBS buffer were used as negative controls.

FIG. 9 shows that JM1-24-3 treatment of the melanoma cells can induce changes in the expression of proteins that are likely involved in downstream signaling. IPA analyses revealed top canonical pathways, top upstream regulators, the molecular and cellular functions of proteins with altered expression levels in melanoma cells treated with either JM1-24-3 full-length antibody or its F(ab′)2 fragments.

FIGS. 10A-10B show that JM1-24-3 blocks the migration and invasion of melanoma A375 cells by neutralization of MUC18. FIG. 10A. Comparison of JM1-24-3 with an irrelevant mAb on the inhibition of cell migration detected with the QCM™ 24-Well Colorimetric Cell Migration Assay Kit showed reduction in migration on treatment with JM1-24-3. FIG. 10B. Comparison of these two mAbs on the invasion of A375 cells using QCM™ ECMatrix Cell Invasion Assay Kit showed that JM1-24-3 treatment reduced invasion of A375 cells.

FIG. 11 shows metastasis studies conducted by injecting 1 million WM266-4 cells into tail vein of athymic nude mice. One day before, mice were pre-treated with JM1-24-3 (n=5) or with irrelevant mAb (n=7) (6 mg/kg/b.wt/i.p/twice a week) following which all received tail vein injection with WM266-4 melanoma cells (1 million). All mice were sacrificed at day 45 and lungs were harvested. Excised lung tissues were further processed for H&E staining. Representative images of H&E staining of the tumor colonies showing small, medium and large lung colonies of melanoma tumor.

FIG. 12 shows a comparison of JM1-24-3 binding with commercially available MUC18 antibody on IHC of patient tissues. JM1-24-3 Ab and mouse anti-human MUC18 Ab (Cat #ab233923; AbCam, Cambridge, MA) was compared for binding on IHC of patient tissues. JM1-24-3 showed better staining intensity and more staining of cancer cells compared to commercial Ab on same dilution (1:1000). Consequently cut tissues were used for staining and same tissue area was compared.

FIG. 13 shows binding of JM1-24-3 to its target on melanoma and other cancer cell surfaces by FACS. On comparing the binding of JM1-24-3 to the surfaces of various cancer cell lines, melanoma cells lines A2058 and WM266-4 showed the strongest signal; gastric cancer (GC) cell line MKN45 had medium binding signals; triple-negative breast cancer (TNBC) cell lines MDA-MB-231 and MDA-MB-468 and the GC cell line BGC823 had weak binding signals. Lymphoma cell lines Ly-3 and LY-10, breast cancer cell lines MDA-MB-453 (Her2/neu++) and MCF-7 (ER+) had no detectable signal (data not shown). Human PBMC served as control.

### DETAILED DESCRIPTION

Therapies targeting common tumor mutations or enhancing anti-tumoral immunity for use in therapeutic methods (e.g., anti-cancer) have been developed. However, there remains a need for further therapeutics which more specifically and effectively target novel mechanisms involved in cancer. Disclosed herein are antibodies capable of targeting MUC18. In particular, the clinical relevance of a novel MUC18 antibody, as well as the structural and functional interactions of the antibody with MUC18, are described. The MUC18 antibodies described can interact with functional, glycosylated MUC18 protein on MUC18+ cancer cells, for example, melanoma cells, to neutralize MUC18 protein and initiate downstream signaling events associated with inhibition of melanoma cell proliferation, migration, and invasion in vitro and reduction in tumor growth and metastasis in vivo. These signaling events depend on binding of the MUC18 antibody to a conformational epitope on the extracellular domain of MUC18.

A novel strategy of live cell immunization and live cell high-throughput screening by FACS analysis was used for the development and screening of antibodies directed against antigens on the surface of metastatic melanoma tumor cells. Multiple protein-, peptide- and cell-based assays were used to define JM1-24-3 as a specific antibody binding to an identified conformational epitope on its target on cancer cells. Mass spectrometry analysis suggested the target as MUC18 (CD146). Evaluation of glycosylation of MUC18 determined that sugar residues were involved in the structure of the molecule, and that functional interactions between JM1-24-3 and the conformational epitope of MUC18 were dependent on glycosylation.

Importantly, JM1-24-3 was capable of binding to MUC18 expressed on the melanoma cell surface, subsequently inducing downstream signaling pathways and further inhibition of cell growth and metastasis. Mechanistic investigations demonstrated induction of multiple changes in downstream signaling pathways following binding of JM1-24-3 to MUC18. Furthermore, JM1-24-3 demonstrated only weak, patchy binding to smooth muscle cells in small vessels of the kidney, lymph nodes and skin, and was otherwise unreactive across a cross-section of normal tissues. This suggests that targeting MUC18 via the conformational epitope identified by JM1-24-3 may have limited toxicity. Finally, in addition to confirming that MUC18 was highly expressed on melanoma tumors, MUC18 was shown to be significantly expressed in several other cancers, including those of the gastrointestinal tract and TNBC.

Although a small number of potentially therapeutic antibodies have been recently developed against MUC18, the specific advantage of JM1-24-3 is that it recognizes a conformational epitope of MUC18 on the cancer cell surface, and in doing so, alters downstream signaling pathways, resulting in reduction of associated tumor-promoting functions that mediate the metastatic phenotype. Thus, the MUC18 conformational epitope may be a promising therapeutic target potentially amenable to mAb treatment. Also, since MUC18 is expressed on the surface of other cancers, JM1-24-3 could be a useful therapeutic agent for therapy of other cancers, either alone or in combination with other agents.

Antibodies of the present disclosure and derivatives thereof (e.g., antibody fragments, antibody-like molecules, etc.) may therefore be useful in the treatment of various conditions including, for example, cancer. In some embodiments, antibodies of the present disclosure are useful in the treatment of MUC18+ cancers, for example, melanoma, osteosarcoma, prostate cancer, ovarian cancer, lung cancer, including non-small cell lung cancer and small-cell lung cancer, breast cancer, including triple negative breast cancer, and gastrointestinal cancer, including hepatocellular carcinoma and pancreatic cancer.

### I. Definitions

“Individual, “subject,” and “patient” are used interchangeably and can refer to a human or non-human.

The terms “lower,” “lowered,” “reduce,” “reduced,” “reduction,” “decrease,” “decreased,” “inhibit,” “inhibited,” or “inhibition” are all used herein generally to mean a decrease by a statistically significant amount. However, for avoidance of doubt, “lower,” “lowered,” “reduce,” “reduced,” “reduction,” “decrease,” “decreased,” “inhibit,” “inhibited,” or “inhibition” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

The terms “increased,” “increase,” “enhanced,” “enhance,” “activated,” or “activate” are all used herein to generally mean an increase by a statically significant amount; for the avoidance of any doubt, “increased,” “increase,” “enhanced,” “enhance,” “activated,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus (N-terminus) and a translation stop codon at the 3′ (carboxy) terminus (C-terminus). A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

Abbreviations used herein include but are not limited to the following: HTS—High-Throughput Screening; MAb—Monoclonal Antibody; MCAM—Melanoma Cell Adhesion Molecule; CD146—Cluster of Differentiation 146; METCAM/MelCAM—Metastatic Melanoma CAM; IACUC—Institutional Animal Care and Use Committee; ATCC—American Type Culture Collection; PBMC—Human Peripheral Blood Mononuclear cells; DMEM—Dulbecco's Modified Eagle's Medium; FFPE—Formalin-Fixed Paraffin Embedded; TMA—Tissue Micro Arrays; s.c.—subcutaneously; i.p.—intraperitoneal; FACS—Fluorescence-activated Cell Sorting; MFI—Mean Fluorescence Intensity; DMSO—Dimethyl sulfoxide; WB—western blot; SDS-PAGE—sodium dodecyl sulfate polyacrylamide gel electrophoresis; ELISA—Enzyme-linked Immunosorbent Assay; FLISA—Fluorescence-linked immunosorbent assay; FITC—Fluorescein isothiocyanate; HRP—Horseradish Peroxidase; Alexa Fluor 647—far-red-fluorescent dye; SNL—Sambucus nigra lectin; DSA—Datura stramonium agglutinin; LCA—Lens culinaris agglutinin; WGA—Wheat germ agglutinin; IHC—Immunocytochemistry; IP—Immunoprecipitation; MS—Mass Spectrometry; RPPA—Reverse Phase Protein Array; IPA—Ingenuity Pathway Analysis; BLI—Bio-Layer Interferometry chips; Fc region—fragment crystallizable region; Fab fragment—fragment antigen-binding; nu/nu—athymic nude mice; SD—standard deviation; Calprotectin—S100 calcium-binding protein A8/A9, S100A8/A9; TLR4—Toll Like Receptor 4; RAGE—Receptor for Advanced Glycation End products; PKB—Protein kinase B; AKT—Serine/threonine-specific kinase; and GCNT3—β-1,3-galactosyl-O-glycosyl-glycoprotein β-1,6-N-acetylglucosaminyltransferase.

### II. MUC18

MUC18, also known as MCAM (melanoma cell adhesion molecule), CD146 (cluster of differentiation 146), METCAM/MelCAM (metastatic melanoma CAM), Mucin 18, MUC18 glycoprotein, Melanoma associated Antigen A32, S-endo 1 Endothelial Associated Antigen, or Melanoma Adhesion Molecule Mel-CM, is a 113 kDa transmembrane glycoprotein encoded by the MCAM gene that belongs to the immunoglobulin superfamily and functions as a calcium-independent adhesion molecule. Two isoforms exist (MCAM long [MCAM-1] and MCAM short [MCAM-s]) which differ in the length of their cytoplasmic domain. Activation of these isoforms may produce functional differences. For example, natural killer cells transfected with MCAM-1 demonstrate decreased rolling velocity and increased cell adhesion to an endothelial cell monolayer and increased microvilli formation while cells transfected with MCAM-s showed no change in adhesion characteristics.

MUC18 is a mediator of several intracellular signaling mechanisms. Expression of MUC18 has been demonstrated to promote tumorigenesis and tumor progression. MUC18 has been studied extensively in malignant melanoma, and its expression in melanoma cell lines has been shown to correlate with the ability of the cells to grow and produce metastases in vivo. Mice with melanoma treated with a fully humanized anti-MCAM/MUC18 antibody (ABX-MA1) developed small tumors at the injection sites and fewer lung metastases than control IgG treated mice. In osteosarcoma, the incidence of spontaneous lung metastases was significantly lower in mice treated with ABX-MA1 compared to IgG-treated control mice. Additionally, therapeutic targeting of MUC18 can reduce bone metastasis in a prostate cancer model.

Circulating MUC18 is a promising melanoma biomarker; circulating levels are significantly associated with poor prognosis and death. MUC18 interacts with several receptor proteins, including Calprotectin (S100 calcium-binding protein A8/A9, S100A8/A9), heparin sulfate, Toll Like Receptor 4 (TLR4) and Receptor for Advanced Glycation End products (RAGE). Thus, MUC18 may play role in multiple processes including inflammation, cell differentiation, adhesion, tumorigenesis, migration, invasion, angiogenesis, and metastasis. MUC18 has been shown to induce translational initiation and transcriptional activation of c-Jun/c-Fos in hepatocellular carcinoma and has been suggested as a potential therapeutic target in malignant rhabdoid tumors through induction of apoptosis by inactivating protein kinase B (PKB) or the serine/threonine-specific kinase (AKT) signaling pathway.

Recent literature suggests that MUC18 can be glycosylated via N-acetyl-glucosaminyltransferease III and V without a role in migration or could be glycosylated and stabilized by β-1,3-galactosyl-O-glycosyl-glycoprotein β-1,6-N-acetylglucosaminyltransferase 3 (GCNT3) playing a major role in melanoma migration and invasion. Thus, involvement of glycosylated conformational epitope in the function of MUC18 could therefore have implications in melanoma progression or metastasis. Recent studies also suggest that aberrant expression of sialic acid (sialylation) plays a major role in cancer cell growth, metastasis and immune evasion, and blocking sialic acid can suppress tumor growth by enhancing T-cell mediated tumor immunity; thus targeting sialic acid is an attractive therapeutic option to overcome immune suppression, here potentially through the epitope on MUC18 recognized by the antibodies disclosed herein.

MUC18 expression is not limited to malignant tissues, as it is also expressed at low levels on several normal adult tissues including smooth muscle, endothelium, mammary ductal, lobular epithelium, and peripheral nerve tissue. Given the physiological expression of MUC18 non-cancer tissues, utilizing it as a target for cancer therapy can present a risk for off-target toxicity and other detrimental side effects. A potential approach to limit off-target toxicity includes engineering antibodies to specifically and effectively recognize cancer-specific MUC18.

An example polypeptide sequence of human MUC18, isoform 1 is provided:

(SEQ ID NO: 24)
MGLPRLVCAFLLAACCCCPRVAGVPGEAEQPAPELVEVEV
GSTALLKCGLSQSQGNLSHVDWFSVHKEKRTLIFRVRQGQ
GQSEPGEYEQRLSLQDRGATLALTQVTPQDERIFLCQGKR
PRSQEYRIQLRVYKAPEEPNIQVNPLGIPVNSKEPEEVAT
CVGRNGYPIPQVIWYKNGRPLKEEKNRVHIQSSQTVESSG
LYTLQSILKAQLVKEDKDAQFYCELNYRLPSGNHMKESRE
VTVPVFYPTEKVWLEVEPVGMLKEGDRVEIRCLADGNPPP
HFSISKQNPSTREAEEETTNDNGVLVLEPARKEHSGRYEC
QGLDLDTMISLLSEPQELLVNYVSDVRVSPAAPERQEGSS
LTLTCEAESSQDLEFQWLREETGQVLERGPVLQLHDLKRE
AGGGYRCVASVPSIPGLNRTQLVNVAIFGPPWMAFKERKV
WVKENMVLNLSCEASGHPRPTISWNVNGTASEQDQDPQRV
LSTLNVLVTPELLETGVECTASNDLGKNTSILFLELVNLT
TLTPDSNTTTGLSTSTASPHTRANSTSTERKLPEPESRGV
VIVAVIVCILVLAVLGAVLYFLYKKGKLPCRRSGKQEITL
PPSRKSELVVEVKSDKLPEEMGLLOGSSGDKRAPGDQGEK
YIDLRH

### III. Antibodies

Aspects of the disclosure relate to MUC18-binding proteins. The terms “polypeptide that specifically binds MUC18” and “MUC18-binding protein” are used interchangeably herein. In some embodiments, the MUC18-binding protein specifically binds to an extracellular domain of MUC18. In some embodiments, the MUC18-binding protein preferentially binds to a glycosylated MUC18 compared to a non-glycosylated MUC18.

In some embodiments, the MUC18-binding protein is an antibody, an antibody-like molecule, or an antigen-binding fragment thereof. In some embodiments, the MUC18-binding protein is an antibody, a nanobody, a minibody, an scFv fragment, or an Fab fragment. In some embodiments, the MUC18-binding protein is a human antibody, humanized antibody, recombinant antibody, chimeric antibody, an antibody derivative, a veneered antibody, a diabody, a monoclonal antibody, or a polyclonal antibody. In some embodiments, the MUC18-binding protein is a monoclonal antibody. In some embodiments, the MUC18-binding protein is a murine antibody. In some embodiments, the MUC18-binding protein is JM1-24-2. In some embodiments, the MUC18-binding protein is a humanized antibody.

In some embodiments, the disclosed MUC18-binding proteins include antibodies comprising a heavy chain variable region (VH) having 85%, 86%, 87%, 88%, 89%, 90%, 92%, 93%, 94%, 95%, 96%, 97%, 98%, 99%, or 100% sequence identity, or any range derivable therein, with SEQ ID NO:20 and a light chain variable region (VL) having 85%, 86%, 87%, 88%, 89%, 90%, 92%, 93%, 94%, 95%, 96%, 97%, 98%, 99%, or 100% sequence identity, or any range derivable therein, with SEQ ID NO:21. In some embodiments, the VH has at least 85% identity with SEQ ID NO:20 and VL has at least 85% identity with SEQ ID NO:21. In some embodiments, the VH has at least 95% identity with SEQ ID NO:20 and the VL has at least 95% identity with SEQ ID NO:21. In some embodiments, the VH comprises SEQ ID NO:20 and the VL comprises SEQ ID NO:21.

The term “antibody” refers to an intact immunoglobulin of any class or isotype, or a fragment thereof that can compete with the intact antibody for specific binding to the target antigen. An isotype refers to the genetic variations or differences in the constant regions of the heavy and light chains of an antibody. In humans, there are five heavy chain isotypes: IgA, IgD, IgG, IgE, and IgM and two light chain isotypes: kappa and lambda. The IgG class is divided into four isotypes: IgG1, IgG2, IgG3 and IgG4 in humans, and IgG1, IgG2a, IgG2b and IgG3 in mice. They share more than 95% homology in the amino acid sequences of the Fc regions but show major differences in the amino acid composition and structure of the hinge region.

The term “antibody” includes a polyclonal antibody, a monoclonal antibody, a chimeric antibody, a human antibody, a veneered antibody, a diabody, a humanized antibody, an antibody derivative, a recombinant antibody, a recombinant humanized antibody, an engineered antibody, a multi-specific antibody, a DARPin, or a derivative or fragment of each thereof. Also contemplated are antibodies having specificity for more than one antigen or target, including bispecific antibodies, trispecific antibodies, tetraspecific antibodies, and other multispecific antibodies.

As used herein, an “antibody” includes whole antibodies and any antigen binding fragment or a single chain thereof. Thus the term “antibody” includes any protein or peptide containing molecule that comprises at least a portion of an immunoglobulin molecule. As used herein, the terms “antibody” or “immunoglobulin” are used interchangeably and refer to any of several classes of structurally related proteins that function as part of the immune response of an animal, including IgM, IgD, IgG, IgA, IgE, and related proteins, as well as polypeptides comprising antibody CDR domains that retain antigen-binding activity. Examples of such include, but are not limited to a complementarity determining region (CDR) of a heavy or light chain or a ligand binding portion thereof, a heavy chain or light chain variable region, a heavy chain or light chain constant region, a framework (FR) region or any portion thereof or at least one portion of a binding protein. In certain embodiments, the antibody or antigen binding fragment specifically binds human MUC18.

In one embodiment, the anti-MUC18 antibody or antigen binding fragment thereof is a neutralizing antibody or antigen-binding fragment thereof. The term “neutralizing” in the context of a MUC18 neutralizing antibody refers to an antibody that may do one or more of: interfere with the MUC18 receptor/MUC18 interaction; reduce the concentration of MUC18 receptor/MUC18 interacted species in a subject or a cell; prevent the MUC18 receptor/MUC18 interaction in a subject or a cell; and/or reduce the biological function of MUC18, which may include, but is not limited to, one or more of: promoting tumorigenesis or tumor progression; promoting cell growth and metastases; promoting inflammation, cell differentiation, adhesion, tumorigenesis, migration, invasion, and angiogenesis; inducing translational initiation and transcriptional activation of c-Jun/c-Fos; or inducing apoptosis by inactivating protein kinase B (PKB) or the serine/threonine-specific kinase (AKT) signaling pathway.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable of binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen would recognize an epitope on the target antigen within a complex mixture.

The epitope regions of a given polypeptide can be identified using many different epitope mapping techniques well known in the art, including: x-ray crystallography, nuclear magnetic resonance spectroscopy, site-directed mutagenesis mapping, protein display arrays, and hydrogen-deuterium exchange see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986), each of which is incorporated by reference herein in their entirety. Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, for chimeric antibodies, the variable regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The tem “variable region” refers to a portion of the antibody that gives the antibody its specificity for binding antigen. The variable region is typically located at the ends of the heavy and light chains. Variable loops of β-strands, three each on the light (VL) and heavy (VH) chains are responsible for binding to the antigen. These loops are referred to as the “complementarity determining regions” (CDRs). In general, the CDR residues are directly and most substantially involved in influencing antigen binding.

The term “constant region” refers to a portion of the antibody that is identical in all antibodies of the same isotype. The constant region differs in antibodies of different isotypes.

The term “light chain” may describe a full-length light chain or fragments thereof. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” may describe a full-length heavy chain or fragments thereof. For example, a full-length heavy chain for human IgG1 has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The isotype of an antibody can be IgM, IgD, IgG, IgA, or IgE and is defined by the heavy chains present of which there are five classifications: mu (μ), delta (d), gamma (γ), alpha (α), or epsilon (ε) chains, respectively. Human IgG has several subtypes, including, IgG1, IgG2, IgG3, and IgG4.

A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one immunoglobulin unit. Monomers are the basic functional units of antibodies. The term “dimer” means an antibody containing two immunoglobulin units attached to one another via constant domains of the antibody heavy chains (the Fc, or fragment crystallizable, region). The complex may be stabilized by a joining (J) chain protein. The term “multimer” means an antibody containing more than two immunoglobulin units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have paratopes (i.e., antigen-binding sites) for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies may be sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as a single chain variable fragment (scFv). Diabodies and scFvs can be constructed without an Fc region, using only variable domains. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies with two antigen-binding sites in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001), each of which is incorporated herein by reference in their entirety).

The part of the Fv fragment of an antibody molecule that binds with high specificity to the epitope of the antigen is referred to herein as the “paratope.” The paratope consists of the amino acid residues that contact the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, framework regions (FRs). The hypervariable loops are the regions of highest primary sequences variability among the antibody molecules from any mammal. The term hypervariable loop is sometimes used interchangeably with the term “complementarity determining region” (CDR). The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1, CDR-L2, and CDR-L3. The L3 (CDR-L3) is generally the region of highest variability among all antibody molecules produced by a given organism. The CDRs are regions of the polypeptide chain arranged linearly in the primary structure and separated from each other by FRs. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1, CDR-H2 and CDR-H3. The majority of amino acid residues in the variable domains, or Fv fragments (VH and VL), are part of the FRs (approximately 85%).

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, J Exp Med, 132(2): 211-50 (1970)); Chothia (as described in C. Chothia et al., Nature, 342(6252): 877-83 (1989)); and IMGT (as described in M.-P. Lefranc et al., Developmental & Comparative Immunology, 27(1): 55-77 (2003)). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding. Depending on the type and size of the antigen, different CDR residues may contact the antigen. See Almagro JC. J Mol Recognit. 17(2):132-43 (2004), incorporated herein by reference.

One skilled in the art can use any of several methods to determine the paratope of an antibody. These methods include:
1) Computational predictions of the tertiary structure of the antibody/epitope binding interactions based on the chemical nature of the amino acid sequence of the antibody variable region and composition of the epitope.
2) Hydrogen-deuterium exchange and mass spectroscopy.
3) Polypeptide fragmentation and peptide mapping approaches in which one generates multiple overlapping peptide fragments from the full length of the polypeptide and evaluates the binding affinity of these peptides for the epitope.
4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof (and/or one or more FRs thereof) that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017), each of which references are incorporated herein by reference in their entirety.

Chimeric immunoglobulins are the products of fused genes derived from different species (the various domains of the antibodies' heavy and light chains are coded for by DNA from more than one species; see, e.g., U.S. Pat. No. 4,816,567); “humanized” antibodies generally have the FRs from human immunoglobulins and one or more CDRs are from a non-human source (e.g., murine).

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region of the recipient are replaced by residues from a variable region of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin, a non-human antibody containing one or more amino acids in a framework region, a constant region or a CDR, that have been substituted with a correspondingly positioned amino acid from a human antibody. In general, humanized antibodies are expected to produce a reduced immune response in a human host, as compared to a non-humanized version of the same antibody. The humanized antibodies may have conservative amino acid substitutions which have substantially no effect on antigen binding or other antibody functions. Conservative substitutions groupings include: glycine-alanine, valine-leucine-isoleucine, phenylalanine-tyrosine, lysine-arginine, alanine-valine, serine-threonine and asparagine-glutamine. Humanization or engineering of antibodies can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

In some embodiments, minimizing the antibody polypeptide sequence from the non-human species optimizes chimeric antibody function and reduces immunogenicity. Specific amino acid residues of the non-human antibody are modified to be homologous to corresponding residues in a human antibody. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. In some instances, corresponding non-human (e.g., murine) residues replace framework region residues of the human immunoglobulin. Replacement of human framework region residues with non-human framework region residues may serve to improve and/or restore antigen binding. Furthermore, humanized antibodies may comprise residues that are not found in the recipient antibody or in the donor antibody to further refine performance. The humanized antibody may also comprise at least a portion of an immunoglobulin constant region, typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988); Almagro et al., Front Immunol 8; 1751 (2018); and Payes et al., Genetic Engineering of Antibody Molecules. In Reviews in Cell Biology and Molecular Medicine. John Wiley & Sons, Inc., Hoboken, New Jersey, USA. 1(3): 1-52 (2015), each of which is incorporated by reference herein in its entirety.

Intrabodies are intracellularly localized immunoglobulins that bind to intracellular antigens as opposed to secreted antibodies, which bind antigens in the extracellular space.

Antibodies also include “linear antibodies.” The procedure for making linear antibodies is known in the art and described in Zapata et al., 1995. Briefly, these antibodies comprise a pair of tandem Ed segments (VH-CH1-VH-CH1) which form a pair of antigen binding regions. Linear antibodies can be bispecific or monospecific.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope. Thus, polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

A monoclonal antibody or “mAb” refers to an antibody obtained from a population of substantially homogeneous antibodies from an exclusive parental cell, e.g., the population is identical except for naturally occurring mutations that may be present in minor amounts. Each monoclonal antibody is directed against a single antigenic determinant (epitope). Monoclonal antibodies are highly specific, as each monoclonal antibody is directed against a single determinant on the antigen.

B. Functional Antibody Fragments and Antigen-Binding Fragments

1. Antigen-Binding Fragments

Certain aspects relate to antibody fragments, such as antibody fragments that bind to antigen. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Holt et al. Trends Biotechnol. 21(11):484-90 (2003)) constituted with a single VH or VL domain; (v) isolated complementarity determining region (CDR) regions. Such terms are described, for example, in Harlow and Lane, Antibodies: A Laboratory Manual, Cold Spring Harbor Laboratory, N Y (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference in their entirety.

Antigen-binding fragments also include fragments of an antibody that retain exactly, at least, or at most 1, 2, or 3 CDRs from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the variable (VL and VH) and the constant (CL and CH1) domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region. The oligomerization domain comprises self-associating α-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

2. Fragment Crystallizable (Fc) Region

An Fc region contains two heavy chain fragments comprising the C_{H}2 and C_{H}3 domains of an antibody. The two heavy chain fragments are held together by two or more disulfide bonds and by hydrophobic interactions of the C_{H}3 domains. The term “Fc polypeptide” as used herein includes native and mutein forms of polypeptides derived from the Fc region of an antibody. Truncated forms of such polypeptides containing a hinge region that promotes dimerization are included.

3. Cell Surface Receptors

Antigen-binding proteins of the present disclosure may be expressed on the surface of a cell. In some embodiments, antigen-binding proteins are cell surface receptors comprising antigen binding domains (e.g., MUC18-binding domains) disclosed herein. In some embodiments, described herein are cell surface receptors comprising a MUC18-binding domain and one or more additional components or domains. Examples of cell surface receptors of the present disclosure include chimeric antigen receptor (CARs) and T-cell receptors (TCRs). A MUC18-specific cell surface receptor may comprise one or more of an antigen binding domain, a signal peptide, an extracellular spacer, a transmembrane domain, a cytoplasmic region, and a linker. In some embodiments, a cell surface receptor of the present disclosure comprises an antigen binding domain having at least 70%, at least 75%, at least 80%, at least 85%, at least 90%, at least 95%, or 100% sequence identity with one or more of SEQ ID NOs:1-21. Cells expressing a MUC18-specific cell surface receptor may be useful in treating one or more MUC18 associated conditions, as described elsewhere herein.

C. Polypeptides with Antibody CDRs & Scaffolding Domains that Display the CDRs

Antigen-binding peptide scaffolds, such as CDRs, are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal nitric oxide synthase (PIN) may also be used.

D. Antibody Binding

The term “selective-binding agent”, “antigen-binding agent”, or “antigen-binding protein” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the inverse of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE).

Antibodies deemed useful in certain embodiments may have an equilibrium dissociation constant of about, at least about or at most about 10-6, 10-7, 10-8, 10-9, 10-10 M, 10-11 M, 10-12 M, or any range derivable therein. These values are reported for antibodies discussed herein and the same assay may be used to evaluate the binding properties of such antibodies. An antibody of the disclosure is said to “specifically bind” its target antigen when the dissociation constant (KD) is about 10-8 M. The antibody specifically binds antigen with “high affinity” when the KD is about 5×10-9 M, and with “very high affinity” when the K_{D} is about 5×10⁻¹² M.

2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cell receptors respond or recognize. Polypeptide epitopes can be formed from both contiguous amino acids and noncontiguous amino acids juxtaposed by tertiary folding of a polypeptide. In some embodiments, an epitope includes at least 3, for example 5-10 amino acids, in a unique spatial conformation.

Epitope specificity of an antibody can be determined in a variety of ways. One approach, for example, involves testing a collection of overlapping peptides of about 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

It also is possible to determine without undue experimentation, whether an antibody has the same specificity as the antibody of this invention by determining whether the antibody being tested prevents an antibody of this invention from binding the protein or polypeptide with which the antibody is normally reactive. If the antibody being tested competes with the antibody of the invention as shown by a decrease in binding by the monoclonal antibody of this invention, then it is likely that the two antibodies bind to the same or a closely related epitope. Alternatively, one can pre-incubate the antibody of this invention with a protein with which it is normally reactive, and determine if the antibody being tested is inhibited in its ability to bind the antigen. If the antibody being tested is inhibited then, in all likelihood, it has the same, or a closely related, epitopic specificity as the antibody of this invention.

3. Modification of Antigen-Binding Domains

It is understood that the antibodies of the present disclosure may be modified, such that they are substantially identical to the antibody polypeptide sequences, or fragments thereof, and still bind the epitopes of the present disclosure. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present disclosure, providing that the variations in the amino acid sequence maintain at least 75%, more preferably at least 80%, at least 90%, at least 95%, at least 96%, at least 97%, at least 98% and most preferably at least 99% sequence identity. In some embodiments, conservative amino acid replacements are contemplated.

The variable region of the antibodies of the present invention can be modified by mutating amino acid residues within the VH and/or VL CDR 1, CDR 2 and/or CDR 3 regions to improve one or more binding properties (e.g., affinity) of the antibody. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

In addition, the antibodies of the invention may be engineered to include modifications within the Fc region to alter one or more functional properties of the antibody, such as serum half-fife, complement fixation, Fc receptor binding, and/or antigen-dependent cellular cytotoxicity. Such modifications include, but are not limited to, alterations of the number of cysteine residues in the hinge region to facilitate assembly of the light and heavy chains or to increase or decrease the stability of the antibody (U.S. Pat. No. 5,677,425) and amino acid mutations in the Fc hinge region to decrease die biological half-life of the antibody (U.S. Pat. No. 6,165,745).

Conservative replacements (also “conservative substitutions” or “conservative amino acid substitutions”) are those that take place within a family of amino acids that possess similar biochemical properties, including charge, hydrophobicity, and size. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). Thus, a conservative replacement may comprise replacement of an amino acid in one family for an amino acid in the same family (e.g., replacement of a lysine with an arginine, replacement of an aspartate for a glutamate, etc.). Alternatively or in addition, amino acid similarity may be determined using a Blocks Substitution Matrix (BLOSUM), such as BLOSUM62 (Henikoff S and Henikoff JG, Proc. Natl. Acad. Sci. U.S.A 89(22): 10915-9 (1992)). In this case, a conservative replacement may be a substitution of amino acids having a non-negative value on a BLOSUM62 matrix. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Certain preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum, Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

It is also contemplated that the antigen-binding domain may be multi-specific or multivalent by multimerizing the antigen-binding domain with VH and VL region pairs that bind either the same antigen (multi-valent) or a different antigen (multi-specific).

E. Enzymatic or Chemical Modification of Antibodies

Additionally, the antibodies of the invention may be enzymatically or chemically modified to produce further derivatives of the antibodies and antigen binding fragments that are described herein. In some embodiments, the term “antibody derivative” includes post-translational modification to the linear polypeptide sequence of the antibody or fragment. The derivatized antibody or fragment thereof may comprise any molecule or substance that imparts a desired property to the antibody or fragment.

The derivatized antibody can comprise, for example, a chemical post-translational modification, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses). In some embodiments, an antibody or fragment thereof is covalently attached to a molecule or substance, such as a labeling moiety or a therapeutic moiety; covalent attachment does not prevent the antibody from generating an anti-idiotypic response. In some embodiments, an antibody or fragment thereof is non-covalently attached to a molecule or substance, such as a labeling moiety or a therapeutic moiety.

Optionally, an antibody or an antigen-binding fragment can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0486525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

a. Post-Translational Modifications

In some embodiments, the antigen-binding protein has or lacks one or more post-translational modifications such as myristoylation, palmitoylation, isoprenylation or prenylation, farnesylation, geranylgeranylation, glypiation, acylation, acetylation, formylation, alkylation, methylation, amide bond formation, amidation at C-terminus, arginylation, polyglutamylation, polyglycylation, butyrylation, glycosylation, glycation, polysialylation, malonylation, hydroxylation, iodination, phosphorylation, adenylylation, propionylation, S-glutathionylation, S-nitrosylation, S-sulfenylation (aka S-sulphenylation), succinylation, sulfation, biotinylation, pegylation, SUMOylation, ubiquitination, neddylation, pupylation, disulfide bridges, or racemization. In other embodiments, the antigen-binding protein has reduced or increased amounts of one or more post-translational modifications as compared to the same antigen-binding protein expressed in the cell that is native to the encoded gene. The reduction or increase may be by at least or at most 25, 50, 60, 70, 80, 90, 100, 150, 200, 250, 300, 350, 400, 450, 500% or more (or any range derivable therein).

In some aspects, contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861, incorporated herein by reference). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the disclosure to obtain PEGylated derivatives of antibodies. See, e.g., EP 0154316 and EP 0401384, incorporated herein by reference. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

b. Conjugates

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, fluorescein isothiocyanate (FITC), rhodamine, dansyl, umbelliferone, dimethyl acridinium ester (DMAE), Texas red, luminol, nicotinamide adenine dinucleotide phosphate (NADPH), and α- or β-galactosidase.

In luminescence or fluoresecence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases. Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

Antibodies may also be coupled to low molecular weight haptens to increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

Antibody conjugates also include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149; and 4,366,241; each incorporated herein by reference. Molecules containing azido groups may also be used to form covalent bonds to proteins through reactive nitrene intermediates that are generated by low intensity ultraviolet light.

Additional suitable conjugated molecules include ribonuclease (RNase), DNase I, an antisense oligonucleotide, an inhibitory RNA molecule such as a siRNA molecule, an immunostimulatory nucleic acid, aptamers, ribozymes, triplex forming molecules, and external guide sequences (e.g., guide RNAs). Aptamers are small nucleic acids ranging from 15-50 bases in length that fold into defined secondary and tertiary structures, such as stern-loops or G-quartets, and can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Ribozymes are nucleic acid molecules that are capable of catalyzing a chemical reaction, either intramolecularly or intermolecularly. Ribozymes typically cleave nucleic acid substrates through recognition and binding of the target substrate with subsequent cleavage. Triplex forming function nucleic acid molecules can interact with double-stranded or single-stranded nucleic acid by forming a triplex, in which three strands of DNA form a complex dependent on both Watson-Crick and Hoogsteen base-pairing. Triplex molecules can bind target regions with high affinity and specificity. The functional nucleic acid molecules may act as effectors, inhibitors, modulators, and stimulators of a specific activity possessed by a target molecule, or the functional nucleic acid molecules may possess a de novo activity independent of any other molecules.

The antibodies of the invention or antigen-binding regions thereof can also be linked to another functional molecule such as another antibody or ligand for a receptor to generate a bi-specific or multi-specific molecule that binds to at least two or more different binding sites or target molecules. Linking of the antibody to one or more other binding molecules, such as another antibody, antibody fragment, peptide or binding mimetic, can be done, for example, by chemical coupling, genetic fusion, or noncovalent association. Multi-specific molecules can further include a third binding specificity, in addition to the first and second target epitope.

The antibodies or fragments thereof of the present invention may be linked to a moiety that is toxic to a cell to which the antibody is bound to form “depleting” antibodies.

The antibodies of the invention may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The antibodies also can be bound to many different carriers. Thus, this invention also provides compositions containing the antibodies and another substance, active or inert. Examples of well-known carriers include glass, polystyrene, polypropylene, polyethylene, dextran, nylon, amylase, natural and modified cellulose, polyacrylamide, agarose, and magnetite. The nature of the carrier can be either soluble or insoluble for purposes of the invention. Those skilled in the art will know of other suitable carriers for binding monoclonal antibodies, or will be able to ascertain such, using routine experimentation.

In some aspects, contemplated are immunoconjugates comprising an antibody or antigen-binding fragment thereof conjugated (e.g., covalently attached) to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing the targeted cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a genetic fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody is conjugated to one or more drug moieties through a linker. The ADC may be prepared by several routes, employing organic chemistry reactions, conditions, and reagents known to those skilled in the art, including: (1) reaction of a nucleophilic group of an antibody with a bivalent linker reagent, to form antibody-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. ADC may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

Examples of ADC known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, “Antibody Carriers of Cytotoxic Agents in Cancer Therapy: A Review,” In: Monoclonal Antibodies '84: Biological and Clinical Applications, A. Pincera et al., (eds.) pp. 475-506) (1985). Both polyclonal antibodies and monoclonal antibodies have been reported as useful in these strategies (Rowland et al., Cancer Immunol. Immunother. 21:183-87 (1986)).

In certain aspects, ADCs include covalent or aggregative conjugates of antibodies, or antigen-binding fragments thereof, with other proteins or peptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912.

(1) Conjugation Methodology

The conjugated agents can be linked to the antibody directly or indirectly, using any of a large number of available methods. Several methods are known in the art for the attachment or conjugation of an antibody to its conjugate moiety (Amon et al., 1985; Hellstrom et al., 1987; Thorpe, 1985; Baldwin et al., 1985; Thorpe et al., 1982).

Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference).

Monoclonal antibodies may also be reacted with an enzyme in the presence of a coupling agent such as glutaraldehyde or periodate.

Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis(p-diazoniumbenzoyl)-ethylenediamine), diisocyanates (such as toluene 2,6-diisocyanate), and bis-active fluorine compounds (such as 1,5-difluoro-2,4-dinitrobenzene).

In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference).

Site-specific attachment of effector or reporter molecules, wherein the reporter or effector molecule is conjugated to a carbohydrate residue in the Fc region has also been disclosed in the literature (O'Shannessy et al. J. Immunol. Methods 99(2):153-61 (1987)).

Bi-specific and multi-specific molecules can be prepared using methods known in the art. For example, each binding unit of the hi-specific molecule can be generated separately and then conjugated to one another. When the binding molecules are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitroberizoic acid) (DTNB), o-phenylenedimaleimide (oRDM), N-succinimidyl-3-(2-pyridyldithio)propionate (SPDP), and sulfosuccinimidyl 4-(N-maleimidomethyl)cyclohaxane-I-carboxylate (sulfo-SMCC) (Karpovsky et al., 1984; Liu et al., 1985). When the binding molecules are antibodies, they can be conjugated by sulfhydryl bonding of the C-terminus hinge regions of the two heavy chains.

F. Proteins

As used herein, a “protein” or “polypeptide” refers to a molecule comprising at least two amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids (e.g., amino acid substitutions) or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, or more contiguous amino acids or nucleic acids, or any range derivable therein, of any of SEQ ID NOs:1-21.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104,105, 106, 107, 108, 109,110, 111, 112, 113, 114,115, 116, 117, 118, 119, 120, 121,122, 123, 124, 125, 126, 127, 128, 129, 130, 131, 132, or 133 (or any derivable range therein) of any of SEQ ID NOs:1-21.

In some embodiments, the protein or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107,108, 109, 110, 111, 112,113, 114, 115, 116, 117, 118, 119, 120, 121, 122,123, 124, 125, 126, 127, 128, 129, 130, 131, 132, or 133 (or any derivable range therein) contiguous amino acids of any of SEQ ID NOs:1-21.

In some embodiments, the polypeptide or protein may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, or 133 (or any derivable range therein) contiguous amino acids that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of any of SEQ ID NOs:1-21.

In some aspects there is a polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111, 112, 113, 114, 115, 116, 117, 118, 119, 120,121, 122, 123, 124, 125, 126, 127, 128, 129, 130, 131, 132, or 133 of any of SEQ ID NOs:1-23 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, or 133 (or any derivable range therein) contiguous amino acids or nucleotides of any of SEQ ID NOs:1-21.

Nucleotide as well as protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide per ml. The concentration of polypeptide in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

1. Sequences

Amino acid sequences from 10 heavy chain variable region CDRs from the MUC18-binding proteins (e.g., antibodies) of the present disclosure are provided in SEQ ID NOs:1-10 as follows and in Table 1: CDR-H1 (SEQ ID NOs:1-3), CDR-H2 (SEQ ID NOs:4-7), CDR-H3 (SEQ ID NOs:8-10).

TABLE 1

| Polypeptide | SEQ ID NO: | Sequence |
|---|---|---|
| CDR-H1 | 1 | GYAFTSYW |
| CDR-H1 | 2 | GYAFTSYWMN |
| CDR-H1 | 3 | YAFTSYWMN |
| CDR-H2 | 4 | IDPFNGYT |
| CDR-H2 | 5 | AIDPFNGYTE |
| CDR-H2 | 6 | WIGAIDPFNGYTEY |
| CDR-H3 | 7 | AR |
| CDR-H3 | 8 | WGGRLYFDY |
| CDR-H3 | 9 | RWGGRLYFDY |
| CDR-H3 | 10 | ARWGGRLYFDY |

Amino acid sequences from 9 light chain variable region CDRs from the MUC 18-binding proteins (e.g., antibodies) of the present disclosure are provided in SEQ ID NOs:11-19 as follows and in Table 1: CDR-L1 (SEQ ID NOs:11-13), CDR-L2 (SEQ ID NOs:14-16), and CDR-L3 (SEQ ID NOs:17-19).

TABLE 2

| Polypeptide | SEQ ID NO: | Sequence |
|---|---|---|
| CDR-L1 | 11 | QDINSF |
| CDR-L1 | 12 | KASQDINSFLS |
| CDR-L1 | 13 | QDINSFLS |
| CDR-L2 | 14 | RAN |
| CDR-L2 | 15 | RANRLVD |
| CDR-L2 | 16 | TLIYRANRLVD |
| CDR-L3 | 17 | LQYDEFP |
| CDR-L3 | 18 | LQYDEFPYT |
| CDR-L3 | 19 | LQYDEFPY |

The amino acid sequence for the full heavy chain variable region of a MUC18-binding protein disclosed herein is provided in SEQ ID NO:20 as follows:

(SEQ ID NO: 20)
MSCKASGYAFTSYWMNWIKERPGQGLEWIGAIDPFNGYTE
YNHKFKDKAILTADNSSSTVYMQLSSLTSEDSAVYYCARW
GGRLYFDYWGOGTTLTVSSAKTTPPSDY.

The amino acid sequence for the full light chain variable region of a MUC18-binding protein disclosed herein is provided in SEQ ID NO:21 as follows:

(SEQ ID NO: 21)
MTQSPSSMYASLGERVTITCKASQDINSFLSWFQQKPGKS
PKTLIYRANRLVDGVPSRFSGSGSGQDYSLTISSLEYED
MGIYYCLQYDEFPYTFGGGTKLEIKRADAAP.

Embodiments of the present disclosure include antigen-binding proteins (e.g., antibodies, antibody-like molecules, or fragments thereof) comprising a heavy chain variable region (VH) having 85%, 86%, 87%, 88%, 89%, 90%, 92%, 93%, 94%, 95%, 96%, 97%, 98%, 99%, or 100% sequence identity, or any range derivable therein, with SEQ ID NO:20 and a light chain variable region (VL) having 85%, 86%, 87%, 88%, 89%, 90%, 92%, 93%, 94%, 95%, 96%, 97%, 98%, 99%, or 100% sequence identity, or any range derivable therein, with SEQ ID NO:21. In some embodiments, the VH has at least 85% identity with SEQ ID NO:20 and VL has at least 85% identity with SEQ ID NO:21. In some embodiments, the VH has at least 95% identity with SEQ ID NO:20 and the VL has at least 95% identity with SEQ ID NO:21. In some embodiments, the VH comprises SEQ ID NO:20 and the VL comprises SEQ ID NO:21.

In some aspects, the disclosure relates to a MUC18-binding protein comprising (a) a VH comprising: (i) a first complementarity determining region (CDR-H1) amino acid sequence having 85%, 86%, 87%, 88%, 89%, 90%, 92%, 93%, 94%, 95%, 96%, 97%, 98%, 99%, or 100% sequence identity, or any range derivable therein, with SEQ ID NO:1, SEQ ID NO:2, or SEQ ID NO:3; (ii) a second CDR (CDR-H2) amino acid sequence having 85%, 86%, 87%, 88%, 89%, 90%, 92%, 93%, 94%, 95%, 96%, 97%, 98%, 99%, or 100% sequence identity, or any range derivable therein, with SEQ ID NO:4, SEQ ID NO:5, or SEQ ID NO:6; and a third CDR (CDR-H3) amino acid sequence having 85%, 86%, 87%, 88%, 89%, 90%, 92%, 93%, 94%, 95%, 96%, 97%, 98%, 99%, or 100% sequence identity, or any range derivable therein, with SEQ ID NO:7, SEQ ID NO:8, SEQ ID NO:9, or SEQ ID NO:10; and (b) a VL comprising: (i) a first CDR (CDR-L1) amino acid sequence having 85%, 86%, 87%, 88%, 89%, 90%, 92%, 93%, 94%, 95%, 96%, 97%, 98%, 99%, or 100% sequence identity, or any range derivable therein, with SEQ ID NO:11, SEQ ID NO:12, or SEQ ID NO:13; (ii) a second CDR (CDR-L2) amino acid sequence having at 85%, 86%, 87%, 88%, 89%, 90%, 92%, 93%, 94%, 95%, 96%, 97%, 98%, 99%, or 100% sequence identity, or any range derivable therein, with SEQ ID NO:14, SEQ ID NO:15, or SEQ ID NO:16; and a third CDR (CDR-L3) amino acid sequence having 85%, 86%, 87%, 88%, 89%, 90%, 92%, 93%, 94%, 95%, 96%, 97%, 98%, 99%, or 100% sequence identity, or any range derivable therein, with SEQ ID NO:17, SEQ ID NO:18, or SEQ ID NO:19.

In some aspects, the disclosure relates to a MUC18-binding protein comprising (a) a VH comprising: (i) a CDR-H1 amino acid sequence having at least 85% identity with SEQ ID NO:1, SEQ ID NO:2, or SEQ ID NO:3; (ii) a CDR-H2 amino acid sequence having at least 85% identity with SEQ ID NO:4, SEQ ID NO:5, or SEQ ID NO:6; and (iii) a CDR-H3 amino acid sequence having at least 85% identity with SEQ ID NO:7, SEQ ID NO:8, SEQ ID NO:9, or SEQ ID NO:10; and (b) a VL comprising: (i) a CDR-L1 amino acid sequence having at least 85% identity with SEQ ID NO:11, SEQ ID NO:12, or SEQ ID NO:13; (ii) a CDR-L2 amino acid sequence having at least 85% identity with SEQ ID NO:14, SEQ ID NO:15, or SEQ ID NO:16; and (iii) a CDR-L3 amino acid sequence having at least 85% identity with SEQ ID NO:17, SEQ ID NO:18, or SEQ ID NO:19.

In some aspects, the disclosure relates to a MUC18-binding protein comprising (a) a VH comprising: (i) a CDR-H1 amino acid sequence having at least 95% identity with SEQ ID NO:1, SEQ ID NO:2, or SEQ ID NO:3; (ii) a CDR-H2 amino acid sequence having at least 95% identity with SEQ ID NO:4, SEQ ID NO:5, or SEQ ID NO:6; and (iii) a CDR-H3 amino acid sequence having at least 95% identity with SEQ ID NO:7, SEQ ID NO:8, SEQ ID NO:9, or SEQ ID NO:10; and (b) a VL comprising: (i) a CDR-L1 amino acid sequence having at least 95% identity with SEQ ID NO:11, SEQ ID NO:12, or SEQ ID NO:13; (ii) a CDR-L2 amino acid sequence having at least 95% identity with SEQ ID NO:14, SEQ ID NO:15, or SEQ ID NO:16; and (iii) a CDR-L3 amino acid sequence having at least 95% identity with SEQ ID NO:17, SEQ ID NO:18, or SEQ ID NO:19.

In some aspects, the disclosure relates to a MUC18-binding protein comprising (a) a VH comprising: (i) a CDR-H1 amino acid sequence comprising SEQ ID NO:1, SEQ ID NO:2, or SEQ ID NO:3; (ii) a CDR-H2 amino acid sequence comprising SEQ ID NO:4, SEQ ID NO:5, or SEQ ID NO:6; and (iii) a CDR-H3 amino acid sequence comprising SEQ ID NO:7, SEQ ID NO:8, SEQ ID NO:9, or SEQ ID NO:10; and (b) a VL comprising: (i) a CDR-L1 amino acid sequence comprising SEQ ID NO:11, SEQ ID NO:12, or SEQ ID NO:13; (ii) a CDR-L2 amino acid sequence comprising SEQ ID NO:14, SEQ ID NO:15, or SEQ ID NO:16; and (iii) a CDR-L3 amino acid sequence comprising SEQ ID NO:17, SEQ ID NO:18, or SEQ ID NO:19.

In some aspects, the disclosure relates to a MUC18-binding protein comprising (a) a VH comprising: (i) a CDR-H1 amino acid sequence comprising SEQ ID NO:1; (ii) a CDR-H2 amino acid sequence comprising SEQ ID NO:4; and (iii) a CDR-H3 amino acid sequence comprising SEQ ID NO:7; and (b) a VL comprising: (i) a CDR-L1 amino acid sequence comprising SEQ ID NO:11; (ii) a CDR-L2 amino acid sequence comprising SEQ ID NO:14; and (iii) a CDR-L3 amino acid sequence comprising SEQ ID NO:17.

2. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines its functional activity, certain amino acid substitutions can be made in a protein sequence and in its corresponding DNA coding sequence, and nevertheless produce a protein with similar or desirable properties.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ nucleic acid sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative” (also “nonconservative”) In some embodiments, a non-conservative substitution affects a function or activity of the polypeptide. In some embodiments, a non-conservative substitution does not affect a function or activity of the polypeptide. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

G. Nucleic Acids

Some aspects are directed to a nucleic acid encoding for a MUC18-binding protein. In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, polymerase chain reaction (PCR) primers or sequencing primers for identifying, analyzing, mutating or amplifying a polynucleotide encoding a polypeptide, antisense oligonucleotides for inhibiting expression of a polynucleotide, and complementary sequences of the foregoing described herein. The nucleic acids can be single-stranded or double-stranded and can comprise RNA and/or DNA nucleotides and artificial variants thereof (e.g., peptide nucleic acids). In some embodiments, a nucleic acid encoding for a MUC18-binding protein comprises SEQ ID NO:22. In some embodiments, a nucleic acid encoding for a MUC18-binding protein comprises SEQ ID NO:23. In some embodiments, a nucleic acid encoding for a MUC18-binding protein comprises SEQ ID NO:22 and SEQ ID NO:23.

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, or at least 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

1. Sequences

The nucleotide sequence encoding for the full heavy chain variable region of a MUC18-binding protein disclosed herein is provided in SEQ ID NO:22 as follows:

(SEQ ID NO: 22)
ATGTCCTGCAAGGCTTCTGGCTACGCCTTTACTAGTTACT
GGATGAACTGGATAAAAGAGAGGCCTGGACAGGGTCTGGA
ATGGATTGGGGCTATTGATCCTTTCAATGGTTATACTGAG
TACAATCATAAGTTCAAGGACAAGGCCATATTGACTGCAG
ACAATTCCTCCAGCACAGTCTACATGCAACTGAGCAGCCT
GACATCTGAGGACTCTGCAGTCTATTACTGTGCAAGATGG
GGGGGACGTCTCTACTTTGACTACTGGGGCCAAGGCACCA
CTCTCACAGTCTCCTCAGCCAAAACGACACCCCCATCTGA
CTATC.

The nucleotide sequence encoding for the full light chain variable region of a MUC18-binding protein disclosed herein is provided in SEQ ID NO:23 as follows:

(SEQ ID NO: 23)
ATGACCCAGTCTCCATCTTCCATGTATGCATCTCTAGGAG
AGAGAGTCACTATCACTTGCAAGGCGAGTCAGGACATTAA
TAGTTTTTTAAGCTGGTTCCAGCAGAAACCAGGGAAATCT
CCTAAGACCCTGATCTATCGTGCAAACAGATTGGTAGATG
GGGTCCCATCAAGGTTCAGTGGCAGTGGATCTGGGCAAGA
TTATTCTCTGACCATCAGCAGCCTGGAGTATGAAGATATG
GGAATTTATTACTGTCTACAGTATGATGAGTTTCCGTACA
CGTTCGGAGGGGGGACCAAGCTGGAAATAAAACGGGCTGA
TGCTGCACCACT.

In some embodiments, the nucleic acid comprises a nucleotide sequence having 85%, 86%, 87%, 88%, 89%, 90%, 92%, 93%, 94%, 95%, 96%, 97%, 98%, 99%, or 100% sequence identity, or any range derivable therein, with SEQ ID NO:22. In some embodiments, the nucleic acid comprises a nucleotide sequence having at least 85% identity to SEQ ID NO:22. In some embodiments, the nucleic acid comprises a nucleotide sequence having at least 95% identity to SEQ ID NO:22. In some embodiments, the nucleic acid comprises SEQ ID NO:22. In some embodiments, the nucleic acid comprises a nucleotide sequence having 85%, 86%, 87%, 88%, 89%, 90%, 92%, 93%, 94%, 95%, 96%, 97%, 98%, 99%, or 100% sequence identity, or any range derivable therein, with SEQ ID NO:23. In some embodiments, the nucleic acid comprises a nucleotide sequence having at least 85% identity to SEQ ID NO:23. In some embodiments, the nucleic acid comprises a nucleotide sequence having at least 95% identity to SEQ ID NO:23. In some embodiments, the nucleic acid comprises SEQ ID NO:23.

2. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, e.g., Romain Studer et al., Biochem. J. 449:581-594 (2013), incorporated herein by reference. For example, the mutation can quantitatively or qualitatively change the biological activity. Examples of quantitative changes include increasing, reducing or eliminating the activity. Examples of qualitative changes include altering the antigen specificity of an antibody.

### IV. Antibody Production

A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745, each incorporated herein by reference (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques.

Unless specified otherwise, the antibodies can be isolated from any suitable biological source, e.g., murine, rat, rabbit, goat, camelid, sheep or canine.

In an example, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (e.g., U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive.

Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after about two to three weeks) for the desired reactivity. Fusion procedures for making hybridomas, immunization protocols, and techniques for isolation of immunized splenocytes for fusion are known in the art.

For example, a hybridoma is produced by fusing a suitable immortal cell line (e.g., a myeloma cell line such as, but not limited to, Sp2/0, Sp2/0-AG14, NSO, NS1, NS2, AE-1, L.5, P3X63Ag8,653, Sp2 SA3, Sp2 MAI, Sp2 SS1, Sp2 SA5, U397, MIA 144, ACT IV, MOLT4, DA-1, JURKAT, WEHI, K-562, COS, RAJI, NIH 313, HL-60, MLA 144, NAMAIWA, NEURO 2A, CHO, PerC.6, YB2/O) or the like, or heteromyelomas, fusion products thereof, or any cell or fusion cell derived there from, or any other suitable cell line as known in the art, with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, preferably the spleen or lymph nodes, of humans or other suitable animals that have been immunized with the antigen of interest. Any other suitable host cell can also be used for expressing-heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as high-performance liquid chromatography (HPLC). Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

Chimeric, humanized or primatized antibodies of the present invention can be prepared based on the sequence of a reference monoclonal antibody prepared using standard molecular biology techniques. DNA encoding the heavy and light chain immunoglobulins can be obtained from the hybridoma of interest and engineered to contain non-reference (e.g., human) immunoglobulin sequences using standard molecular biology techniques. For example, to create a chimeric antibody, the murine variable regions can be linked to human constant regions using methods known in the art (U.S. Pat. No. 4,816,567). To create a humanized antibody, the murine CDR regions can be inserted into a human framework using methods known in the art (U.S. Pat. Nos. 5,225,539 and 5,530,101; 5,585,089; 5,693,762 and 6,180,370). Similarly, to create a primatized antibody the murine CDR regions can be inserted into a primate framework using methods known in the art (WO 93/02108 and WO 99/55369).

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel et al., 2000; Gallo et al., 2000; Green, 1999; Yang et al., 1999A; Yang, 1999B; Jakobovits, 1998; Green and Jakobovits, 1998; Jakobovits, 1998; Tsuda et al., 1997; Sherman-Gold, 1997; Mendez et al., 1997; Jakobovits, 1996; Jakobovits, 1995; Mendez et al, 1995; Jakobovits, 1994; Arbones et al., 1994; Jakobovits, 1993; Jakobovits et al., 1993; U.S. Pat. No. 6,075,181).

Alternatively, the antibodies of this invention can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species antibodies. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological interest, 4th ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al., 1991.

Other suitable methods of producing or isolating antibodies of the requisite specificity can be used, including, but not limited to, methods that select recombinant antibody from a peptide or protein library (e,g., but not limited to, a bacteriophage, ribosome, oligonucleotide, cDNA, or the like, display library; e.g., as available from various commercial vendors such as MorphoSys (Martinsreid/Planegg, Del.), BioInvent (Lund, Sweden), Affitech (Oslo, Norway) using methods known in the art. Art known methods are described in the patent literature some of which include U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice) (Nguyen et al., 1977; Sandhu et al., 1996); Eren et al., 1998), that are capable of producing a repertoire of human antibodies, as known in the art and/or as described herein. Such techniques, include, but are not limited to, ribosome display (Wanes et al., 1997; Hanes et al., 1998); single cell antibody producing technologies (e,g., selected lymphocyte antibody method (“SLAM”) (U.S. Pat. No. 5,627,052, Wen et al., 1987; Babcook et al., 1996); gel microdroplet and flow cytometry (Powell et al., 1990; Gray et al., 1995; Kenny et al., 1995); B-cell selection (Steenbakkers et al., 1994).

The antibodies of this invention can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

The immunogenicity of a particular immunogen composition can be enhanced by the use of non-specific stimulators of the immune response, known as adjuvants. Adjuvants that may be used in accordance with embodiments include, but are not limited to, interleukin-1 (IL-1), IL-2, IL-4, IL-7, IL-12, γ-interferon (INF-7), granulocyte-macrophage colony-stimulating factor (GMCSF), Bacillus Calmette-Gudrin (BCG), aluminum hydroxide, muramyl dipeptide (MDP) compounds, muramyl tripeptide phosphatidyl ethanolamine (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, NJ), cytokines such as INF-β, IL-2, or IL-12, or genes encoding proteins involved in immune helper functions, such as B7-1 (CD80) or B7-2 (CD86). A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

B. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different VL- and VH-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by N-terminal and/or C-terminal deletions, where the remaining amino acid sequence is substantially identical to the corresponding positions in the naturally occurring sequence deduced, for example, from a full-length cDNA sequence.

Also contemplated herein are non-peptide compounds having properties analogous to those of a template peptide. These types of non-peptide compounds are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987).

Also contemplated are “antibody like binding peptidomimetics” (ABiPs), which are peptide-like molecules that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the disclosure are proteins that are structurally similar to an antibody displaying a desired biological activity, such as the ability to bind a protein, but have one or more peptide linkages optionally replaced by a linkage selected from: —CH2NH—, —CH2S—, —CH2-CH2-, —CH—CH (cis and trans), —COCH2-, —CH(OH)CH2-, and —CH2SO— by methods well known in the art. Systematic substitution of one or more amino acids of a consensus sequence with a D-amino acid of the same type (e.g., D-lysine in place of L-lysine) may be used in certain embodiments of the disclosure to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### V. Obtaining Antibodies

In some aspects, there are nucleic acid molecules encoding antibody or antibody-like polypeptides (e.g., heavy or light chain, variable domain only, or full-length). These may be generated by methods known in the art, e.g., isolated from B cells of mice that have been immunized and isolated, phage display, expressed in any suitable recombinant expression system and allowed to assemble to form antibody molecules.

A. Expression

The nucleic acid molecules may be used to express large quantities of recombinant antibodies or to produce chimeric antibodies, single chain antibodies, antigen-binding fragments, immunoadhesins, diabodies, bi-specific antibodies, mutated antibodies, and other antibody derivatives. If the nucleic acid molecules are derived from a non-human, non-transgenic animal, the nucleic acid molecules may be used for antibody humanization.

1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and/or probes thereof. In addition to control sequences that govern transcription and translation, vectors and expression vectors may contain nucleic acid sequences that serve other functions as well.

To express the antibodies, or antigen-binding fragments thereof, DNA encoding partial or full-length light and heavy chains are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

2. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include, but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide using an appropriate expression system.

3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (U.S. Pat. No. 5,789,215, incorporated herein by reference); by electroporation (U.S. Pat. No. 5,384,253, incorporated herein by reference); by calcium phosphate precipitation; by using DEAE dextran followed by polyethylene glycol; by direct sonic loading; by liposome mediated transfection; by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (U.S. Pat. Nos. 4,684,611 and 4,952,500, each incorporated herein by reference); by desiccation/inhibition mediated DNA uptake. Other methods include viral transduction, such as gene transfer by lentiviral or retroviral transduction.

4. Host Cells

In another aspect, contemplated are the use of host cells into which a polypeptide, nucleic acid, or recombinant expression vector has been introduced. Antibodies and antibody-like molecules can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to immune cell (e.g., T-cell) activation. Control of antibody expression allows immune cells, such as tumor-targeting immune cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors and their cognate polypeptides. Host cells which may be used to express antibodies and other antigen-binding proteins of the present disclosure include, for example, murine myeloma cells (e.g., NS0 cells, SP2/0-Agl4 cells), Chinese hamster ovary (CHO) cells, baby hamster kidney (BHK21) cells, human embryonic kidney 293 (HEK293) cells, fibrosarcoma HT-1080 cells, and PER.C6 cells. In some embodiments, the cell is an immune cell. In some embodiments, the immune cell is a T cell. In some embodiments, the immune cell is a B cell.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

In some embodiments, the cells disclosed herein can be used in methods to generate the MUC18-binding proteins of the disclosure. The methods can comprise culturing the cell under conditions sufficient to express a nucleic acid encoding for the MUC18-binding protein in the cell. Also contemplated are methods for generating the MUC18-binding proteins of the disclosure, the method comprising (a) providing a nucleic acid encoding for the polypeptide to a cell, and (b) subjecting the cell to conditions sufficient to express the polypeptide from the nucleic acid.

B. Isolation

The nucleic acid molecule encoding either or both of the entire heavy and light chains of an antibody or the variable regions thereof may be obtained from any source that produces antibodies. Methods of isolating mRNA encoding an antibody are well known in the art. The sequences of human heavy and light chain constant region genes are also known in the art. Nucleic acid molecules encoding the full-length heavy and/or light chains may then be expressed in a cell into which they have been introduced and the antibody isolated.

### VI. Methods of Treatment and Administration of Therapeutic Compositions

The therapy provided herein may comprise administration of a therapeutic agent (e.g., a MUC18-binding protein). In some embodiments, therapy provided herein comprises administration of a MUC18-binding protein, a nucleic acid encoding for the MUC18-binding protein, a vector comprising the nucleic acid encoding for the MUC18-binding protein, or a cell comprising the MUC18-binding protein, a nucleic acid encoding for the MUC18-binding protein, or a vector comprising the nucleic acid encoding for the MUC18-binding protein, and a pharmaceutically acceptable excipient. In some embodiments, therapy provided herein comprises administration of a combination of therapeutic agents, such as a MUC18-protein and an additional therapeutic agent. The therapy or therapies may be administered in any suitable manner known in the art. For example, for a combination therapy, the MUC18-binding protein, and the additional therapeutic agent may be administered sequentially (at different times) or concurrently (at the same time). In some embodiments, the MUC18-binding protein and the additional therapeutic agent are administered in a separate composition. In some embodiments, the MUC18-binding protein and the additional therapeutic agent are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

An effective amount of therapeutic or prophylactic composition is determined based on the intended goal. The treatments may include various “unit doses.” The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, and the particular route and formulation is within the skill of determination of those in the clinical arts. A unit dose need not be administered as a single injection but may comprise continuous infusion over a set period of time. In some embodiments, a unit dose comprises a single administrable dose.

An effective dose is understood to refer to an amount necessary to achieve a particular effect. In the practice in certain embodiments, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or μM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

A. General Pharmaceutical Compositions

In some embodiments, pharmaceutical compositions are administered to a subject. Different aspects may involve administering an effective amount of a composition to a subject. In some embodiments, an antibody or antigen binding fragment capable of binding to MUC18 may be administered to the subject to protect against or treat a condition (e.g., cancer). Alternatively, an expression vector encoding one or more such antibodies or polypeptides or peptides may be given to a subject as a preventative treatment. Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

B. Methods of Treatment

Compositions (e.g., antigen-binding proteins) or methods described herein may be administered to any patient having a condition in which targeting MUC18 may have therapeutic benefit. Conditions in which targeting MUC18 may have a therapeutic benefit include, for example, a condition associated with activation of MUC18 (e.g., cancer) and a condition in which targeting MUC18 may be used to specifically deliver a therapeutic. Such conditions include, for example, cancer.

The term “treatment” or “treating” means any treatment of a disease in a mammal, including:
(i) preventing the disease, that is, causing the clinical symptoms of the disease not to develop by administration of a protective composition prior to the induction of the disease;
(ii) suppressing the disease, that is, causing the clinical symptoms of the disease not to develop by administration of a protective composition after the inductive event but prior to the clinical appearance or reappearance of the disease;
(iii) inhibiting the disease, that is, arresting the development of clinical symptoms by administration of a protective composition after their initial appearance; and/or
(iv) relieving the disease, that is, causing the regression of clinical symptoms by administration of a protective composition after their initial appearance.

1. Treatment of Cancer

Disclosed herein, in some embodiments, are methods for treating cancer comprising providing to a subject in need thereof a MUC18-binding protein disclosed herein. The cancer may be a solid tumor, metastatic cancer, or non-metastatic cancer. In certain embodiments, the cancer may originate in the bladder, blood, bone, bone marrow, brain, breast, urinary, cervix, esophagus, duodenum, small intestine, large intestine, colon, rectum, anus, gum, head, kidney, liver, lung, nasopharynx, neck, ovary, prostate, skin, stomach, testis, tongue, or uterus.

The cancer may specifically be of one or more of the following histological types, though it is not limited to these: undifferentiated carcinoma, bladder, blood, bone, brain, breast, urinary, esophageal, thymomas, duodenum, colon, rectal, anal, gum, head, kidney, soft tissue, liver, lung, nasopharynx, neck, ovary, prostate, skin, stomach, testicular, tongue, uterine, thymic, cutaneous squamous-cell, noncolorectal gastrointestinal, colorectal, melanoma, Merkel-cell, renal-cell, cervical, hepatocellular, urothelial, non-small cell lung, head and neck, endometrial, esophagogastric, small-cell lung mesothelioma, ovarian, esophogogastric, glioblastoma, adrencorical, vueal, pancreatic, germ-cell, giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; thymoma; thecoma; androblastoma; sertoli cell carcinoma; malignant melanoma; amelanotic melanoma; superficial spreading melanoma; epithelioid cell melanoma; sarcoma; mesenchymal (e.g., fibrosarcoma; fibrous histiocytoma; myxosarcoma; liposarcoma; leiomyosarcoma; rhabdomyosarcoma; embryonal rhabdomyosarcoma; alveolar rhabdomyosarcoma; stromal sarcoma; mullerian mixed tumor; nephroblastoma; hepatoblastoma; carcinosarcoma; dysgerminoma; embryonal carcinoma; choriocarcinoma; mesonephroma; hemangiosarcoma; Kaposi's sarcoma; lymphangiosarcoma; osteosarcoma; juxtacortical osteosarcoma; chondrosarcoma; mesenchymal chondrosarcoma; giant cell tumor of bone; Ewing's sarcoma; ameloblastic odontosarcoma; ameloblastic fibrosarcoma; chordoma; ependymoma; astrocytoma; protoplasmic astrocytoma; fibrillary astrocytoma; astroblastoma; oligodendroglioma; oligodendroblastoma; primitive neuroectodermal; cerebellar sarcoma; ganglioneuroblastoma; neuroblastoma; retinoblastoma; olfactory neurogenic tumor; neurofibrosarcoma; paragranuloma); or hematopoietic (e.g., multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia). In some embodiments, the cancer is a skin cancer. In some embodiments, the cancer is melanoma. In some embodiments, the cancer is a breast cancer. In some embodiments, the breast cancer is triple negative breast cancer. In some embodiments, the cancer is a gastric, or gastrointestinal, cancer. In some embodiments, the gastric cancer is gastrointestinal adenocarcinoma.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

The selected treatment regimen can be an aggressive one which is expected to result in the best clinical outcome (e.g., complete cure of the disease) or a more moderate one which may relieve symptoms of the disease yet results in incomplete cure of the disease. The type of treatment can include a surgical intervention, administration of a therapeutic drug such as a MUC18-binding protein, immunotherapy, an exposure to radiation therapy, and/or any combination thereof. The dosage, schedule and duration of treatment can vary, depending on the severity of disease and the selected type of treatment, and those of skill in the art are capable of adjusting the type of treatment with the dosage, schedule and duration of treatment.

Biomarkers like MUC18 that can predict the efficacy of certain therapeutic regimen and can be used to identify patients who will receive benefit of a conventional single or combined modality therapy before treatment begins or to modify or design a future treatment plan after treatment. In the same way, those patients who do not receive much benefit from such conventional single or combined modality therapy and can offer them alternative treatment(s) may be identified.

C. Kits

Certain aspects of the present disclosure also concern kits containing compositions of the disclosure or compositions to implement methods of the disclosure. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer.

It is contemplated that any method or composition described herein can be implemented with respect to any other method or composition described herein and that different embodiments may be combined. The claims originally filed are contemplated to cover claims that are multiply dependent on any filed claim or combination of filed claims.

### VII. Examples

The following examples are included to demonstrate certain embodiments of the disclosure. It should be appreciated by those of skill in the art that the techniques disclosed in the examples which follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the spirit and scope of the disclosure. The Examples should not be construed as limiting in any way. The contents of all cited references (including literature references, issued patents, published patent applications, and GenBank Accession numbers as cited throughout this application) are hereby expressly incorporated by reference. When definitions of terms in documents that are incorporated by reference herein conflict with those used herein, the definitions used herein govern.

### Example 1—Screening and Identification of Anti-MUC18 mAb

To obtain mAbs against the conformational epitopes of cell surface antigens, the inventors used a strategy of live-cell immunization using a mixture of three melanoma cell lines, including A375 (primary cell line with low metastatic capacity), A2058 (highly metastatic cell line from lymph node), and WM266-4 (highly metastatic cell line from lymph node) as immunogens.

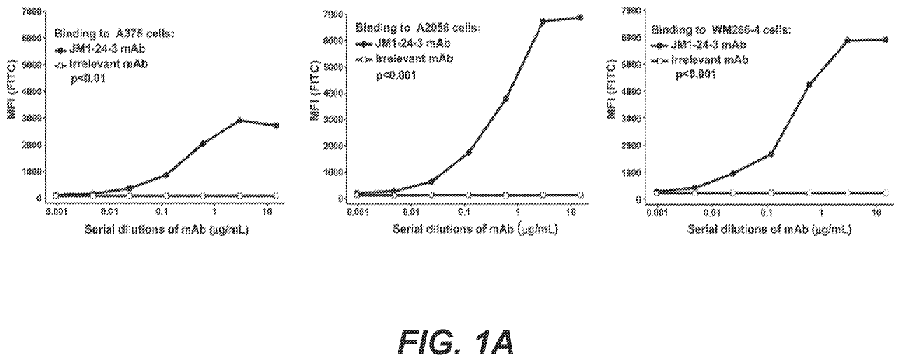

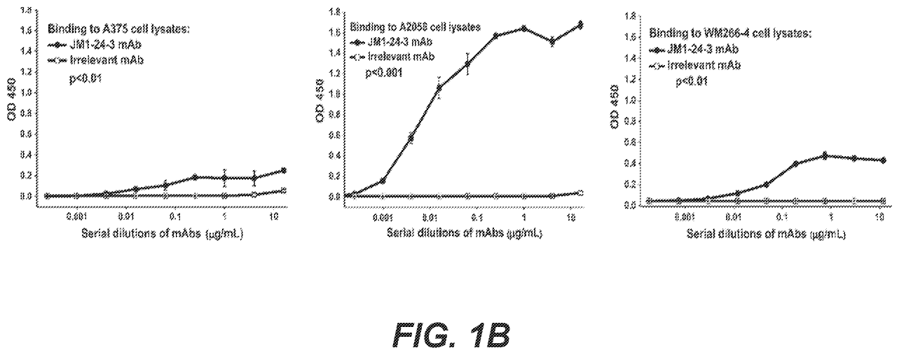

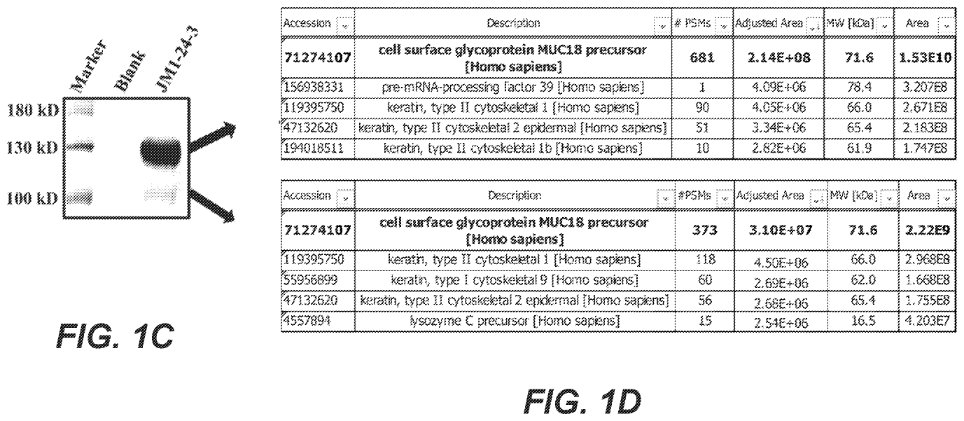

By FACS-HTS, hybridoma supernatant (the hybridoma colony number is about 23,000) binding with the three melanoma cell lines versus normal human PBMC cells was assessed (data not shown). Of the supernatants tested, less than 5% demonstrated binding to the immunization cell lines but no binding to the human PBMC cells. Based on stronger binding signals in FACS assay, 20 promising hybridoma candidates were selected and subcloned, and again tested for binding. Out of those 20 candidates, given its strong binding as measured by FACS assay, JM1-24-3 hybridoma clone was chosen for further evaluation and validation. JM1-24-3 hybridoma was further subcloned, and secreted mAb was purified and identified as an IgG1 heavy chain and κ light chain antibody by mouse antibody isotyping [1]. FACS analysis of binding of JM1-24-3 to each of the immunization cell lines was done (FIG. 1A). Highly metastatic cell lines WM266-4 and A2058 showed greater binding in terms of MFI compared to low metastatic cell line A375 (p<0.01). Similar relative binding patterns were seen with ELISA. Highly metastatic cell line A2058 demonstrated maximum binding (1.67±0.04) at 1.0 μg/mL optimal concentration, while another highly metastatic cell line, WM266-4, demonstrated measurably lower binding (0.43±0.02), and the low metastatic potential A375 cells showed the lowest binding (0.25±0.02) (p<0.01) (FIG. 1B). Thus, FACS-HTS of hybridomas showed differential binding of JM-1-24-3 with low and high metastatic melanoma cells.

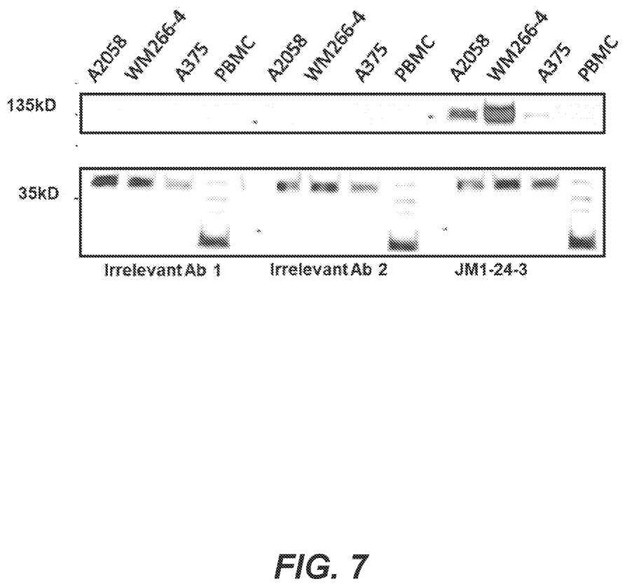

JM1-24-3 was also shown to interact with the glycosylated MUC18 protein expressed on melanoma cells. To identify the JM1-24-3 target on melanoma cells, lysates of A2058, WM266-4 and A375 (25 μg) were immunoprecipitated individually with 1 μg each of JM1-24-3 and other irrelevant Abs followed by SDS-PAGE. One stronger band at 135 kD alone was observed on probing with the same JM1-24-3 mAb, while no band was observed with other irrelevant antibodies. PBMC served as lysate control and j-actin (42 kD) served as loading control (immunoprecipitation [IP] shown in FIG. 7). Further, for mass spectrometry analysis, WM266-4 cells (100 μg) were immunoprecipitated with JM1-24-3 mAb (10 μg) alone. Due to increased lysate loading, two bands (one stronger band at 135 kD and one faint band at 110 kD) were identified in SDS-PAGE Coomassie staining (FIG. 1C). These bands were individually analyzed with mass spectrometry (MS) (FIG. 1D), which indicated that both bands matched the molecular weights of the MUC18 protein as reported previously. The inventors assumed that the two bands observed with two different molecular weights could be because of partial reduction of the MUC18 molecule due to protein denaturation and/or the presence of glycoproteins, rather than from dimer disassociation. Subsequently, the interaction between JM1-24-3 and its target was measured with Octet RED384 (ForteBio, LLC, Fremont, CA) on flow-through Bio-Layer Interferometry (BLI) chips with an average affinity constant K_{D}1.60E-09.

### Example 2—Identifying the Carbohydrate Moieties and Defining the Conformational Epitope of MUC18

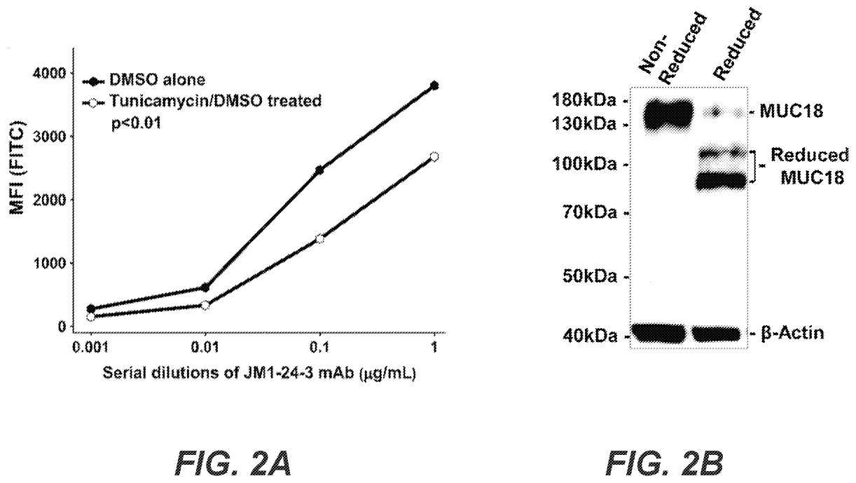

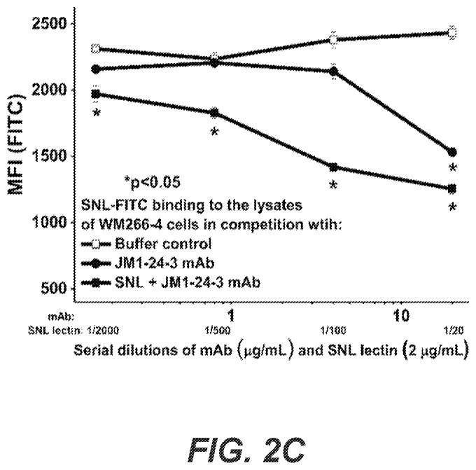

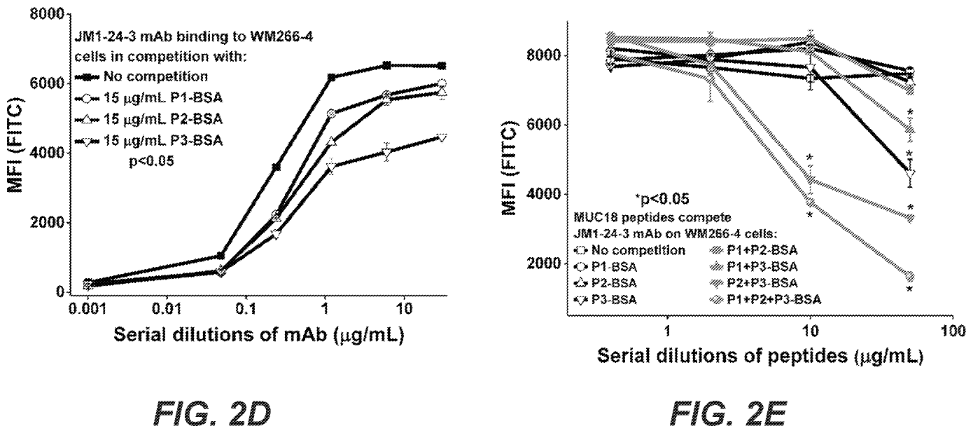

To evaluate the potential functional importance of the carbohydrate moieties in MUC18 and JM1-24-3 interaction sites, tunicamycin was used (FIGS. 2A-2E). Tunicamycin treatment showed significant reduction (40.7±7.47%; p<0.01) in binding of JM1-24-3 to WM266-4 by FACS assay (FIG. 2A). These results suggested that tunicamycin partially digested the carbohydrates in N-linked glycosylation sites resulting in reduction of JM1-24-3 binding as demonstrated in Western blot (FIG. 2B). On tunicamycin treatment, MUC18 showed reduced forms of approximately 120 kDa and 90 kDa.

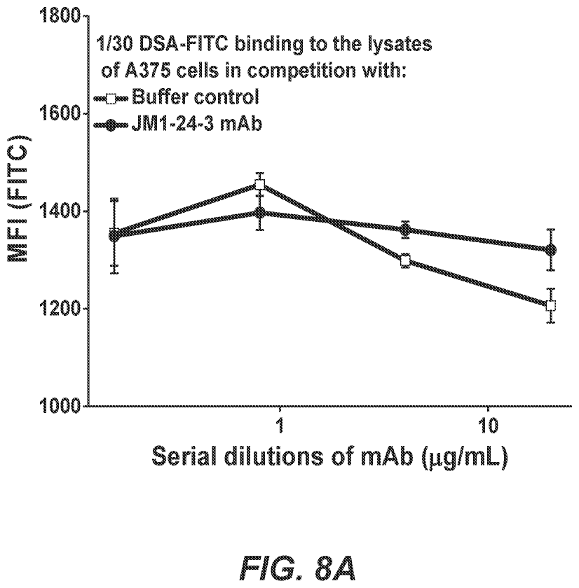

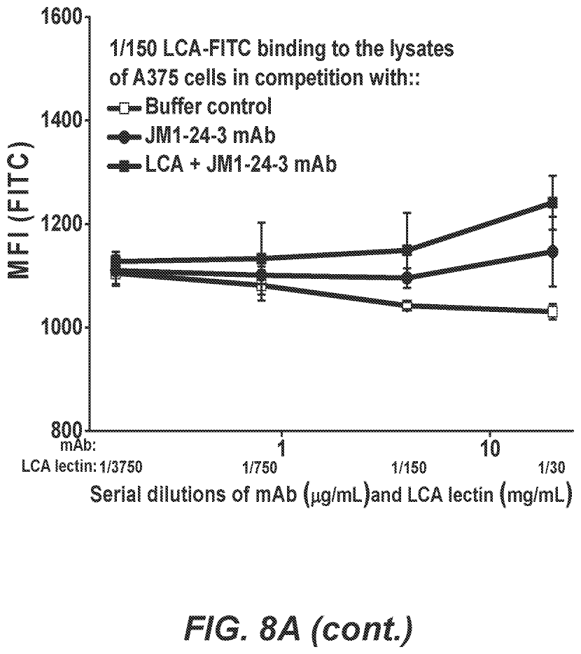

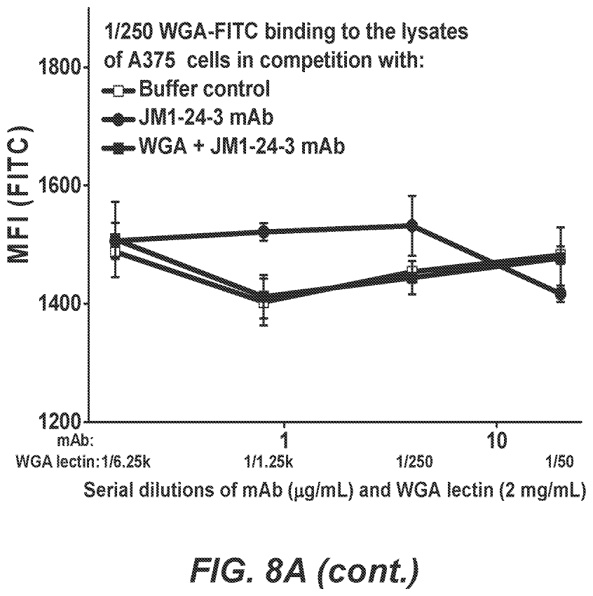

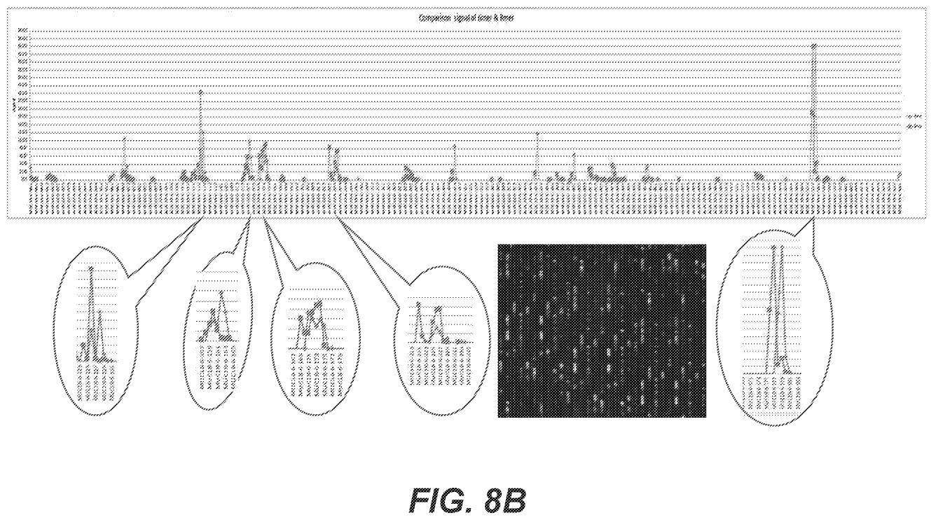

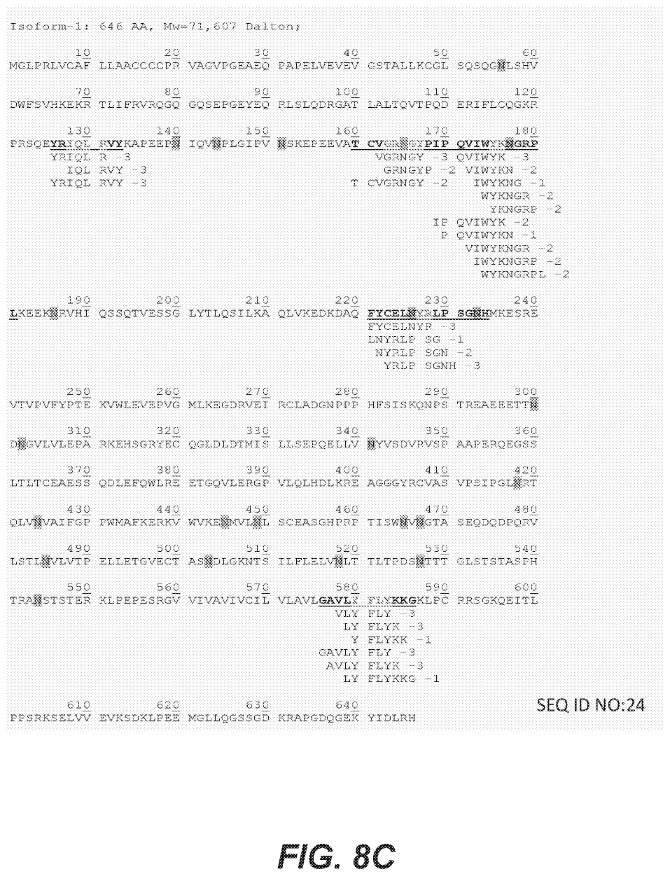

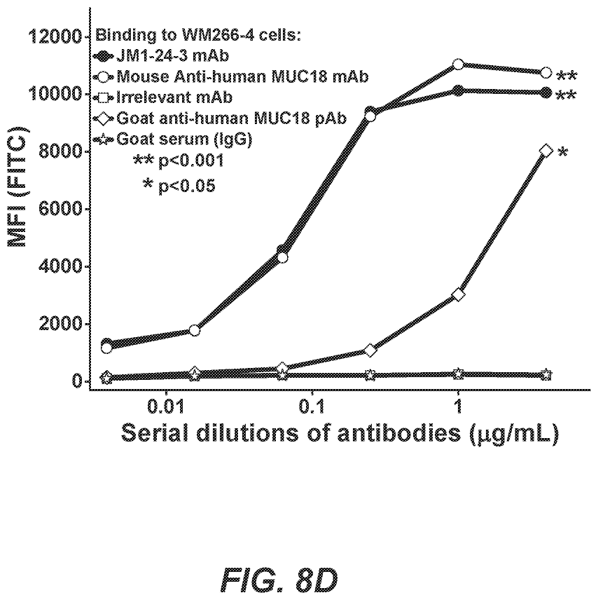

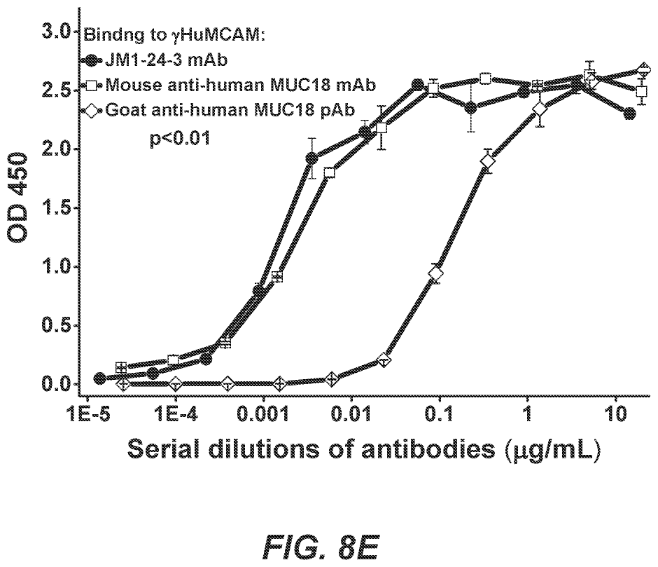

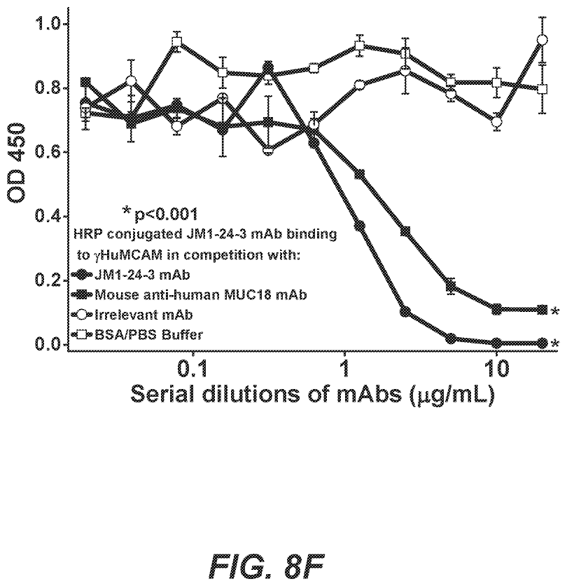

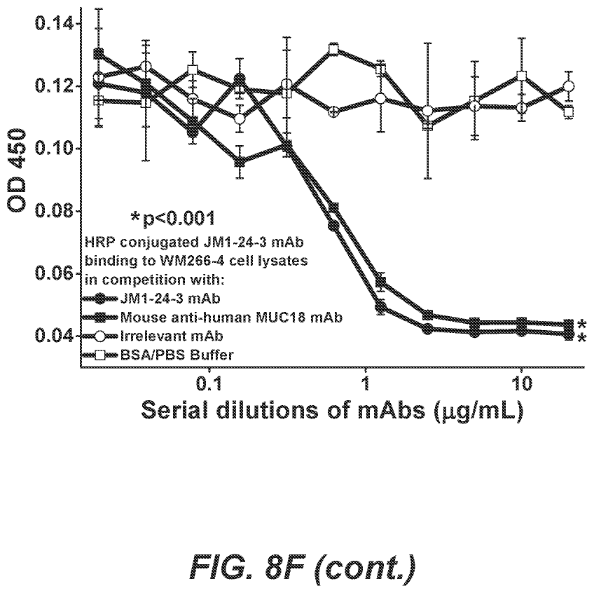

The inventors also investigated the carbohydrate moiety(ies) of MUC18 using FLISA to elucidate key asparagine sites of N-linked glycosylation and evaluated the potential role of carbohydrate chains in the MUC18 epitope interacting with JM1-24-3. The binding of FITC conjugated SNL lectin to MUC18 on WM266-4 and competition with JM1-24-3 or SNL plus JM1-24-3 were quantified by MFI (FIG. 2C). In parallel experiments, JM1-24-3 did not compete with DSA-FITC, LCA-FITC or WGA-FITC (FIG. 8A) for binding to the lysates of A375 cells under identical conditions in FLISA. SNL binds preferentially to sialic acid attached to terminal galactose in a-2,6 and to a lesser degree, a-2,3 linkage.

The epitope of MUC18 interacting with JM1-24-3 was inferred to represent a conformational, N-linked glycosylation site, and the glycoprotein was determined to most likely be the lectin SNL. The inventors next evaluated JM1-24-3 binding to the epitope(s) of MUC18 through oligopeptide microarray (epitope mapping on biochips). The 8-mer and 6-mer tiling oligopeptides with one amino acid resolution derived from human MUC18 protein sequence were synthesized in situ (in triplicates) on microarray chip(s) and JM1-24-3 binding signals (MFI) were detected using anti-mouse IgG Alexa-647 secondary antibody. The binding peaks were analyzed using ArrayPro data processing software (FIG. 8B). Reproducible results from this epitope mapping demonstrated binding peptides as peaks (signals) that aligned with specific MUC18 amino acid sequences (FIG. 8C). Since the binding sites were sporadically spaced with different signal strength a conformational epitope/binding site for JM1-24-3 was suggested, with involvement of multiple MUC18 asparagine (N, Asn) residues.

Further, the inventors validated JM1-24-3 binding to MUC18 using the three peptides selected from the identified epitope binding regions and showed their binding was competitively reduced by each of the MUC18 peptides (BSA conjugated P1-BSA, P2-BSA and P3-BSA), although to different degrees (FIG. 2D). To further confirm that these three peptides in combination could interfere with JM1-24-3 binding to the epitope of MUC18 on melanoma cells, 3 μg/mL of JM1-24-3 bound to WM266-4 cells was subjected to individual and combinatorial binding competition using serial dilutions of selected peptides (FIG. 2E). The competitive interference achieved with two or three peptides in combination was greater than that achieved with the peptides individually.

In order to evaluate the specificity of JM1-24-3 against MUC18, the inventors compared the binding characteristics of JM1-24-3 with two commercially available antibodies directed against MUC18: mouse anti-human MUC18 mAb and goat polyclonal Ab together with an isotype control irrelevant mAb and goat serum (IgG). Representative flow cytometric analyses of MUC18 expression on the surface of WM266-4 cells is shown (FIG. 8D). JM1-24-3 had a binding potency similar to that of the mouse anti-human MUC18 mAb. Similar results were obtained with another form of MUC18, a recombinant human MCAM protein (7HuMCAM), in indirect ELISA analysis (FIG. 8E). The inventors further tested the binding motif and potency of HRP-conjugated JM1-24-3 binding to γHuMCAM and WM266-4 lysate on ELISA, which were individually subjected to competition by JM1-24-3 (self-competition), mouse anti-human MUC18 mAb, irrelevant mAb and BSA/PBS buffer (FIG. 8F). HRP-conjugated JM1-24-3 could be competed, to different extents, by JM1-24-3 (self-competition) and mouse anti-human MUC18 mAb in binding to both the γHuMCAM and WM266-4 lysates. These results demonstrated that JM1-24-3 had higher binding potency than the commercial mouse anti-human MUC18 mAb, and that these two mAbs did not share the same binding site on MUC18. In summary, the binding patterns for the two mAbs were similar in FACS and indirect ELISA, but not in competition ELISA and WB, suggesting they shared part, but not all, of their MUC18 binding epitope.

### Example 3—JM1-24-3 Binding to MUC18 Glycoprotein on Melanoma Cell Surface Activates Downstream Signal Transduction Pathway Involved in Cell Growth and Proliferation

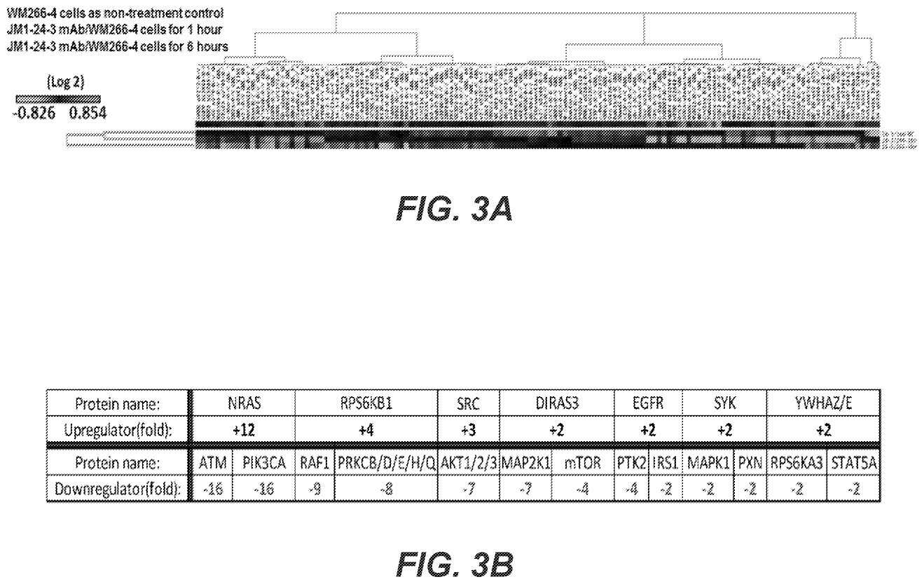

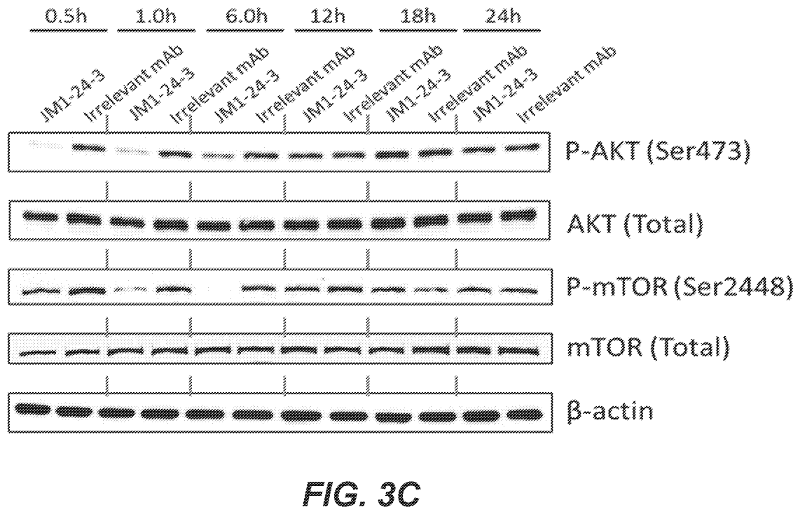

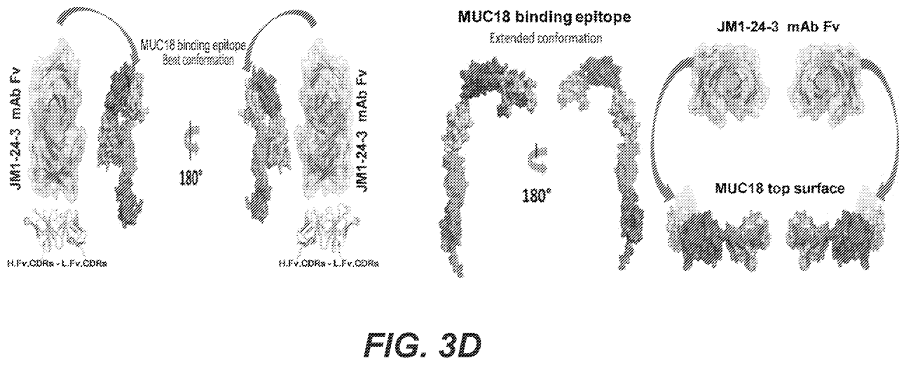

The inventors sought to identify targets and downstream signaling events resulting from the binding of JM1-24-3 to MUC18 by RPPA. Both full length JM1-24-3 and its F(ab′)2 fragments (to avoid the background caused by the secondary goat anti-mouse IgG Fc pAb-FITC) reacted with MUC18 expressed on melanoma cells to induce down-stream signaling in tissue culture. WM266-4 cells were treated with full-length JM1-24-3 at two time points (1 hour and 6 hours) in tissue culture; the lysates from each time point were individually analyzed by RPPA. The heat maps in FIG. 3A demonstrated that approximately 150 signaling proteins were differentially expressed following JM1-24-3 treatment. By comparing with cell lysate without any treatment, several clusters of proteins showed elevated expression levels and a few others tended to have lower expression levels upon JM1-24-3 treatment. JM1-24-3 F(ab′)2 fragments revealed similar expression pattern in RPPA assay as well (data not shown). RPPA data was analyzed using IPA software (FIG. 3B). After JM1-24-3 interacted with MUC18 on WM266-4 cell surface, NRAS, RPS6Kβ1, and SRC were up-regulated, whereas ATM, PIK3CA, RAF1, PRKCB/D/E/H/Q, AKT1/2/3, MAP2K1, mTOR and PTK2 were down-regulated. To validate these RPPA data, WM266-4 cells were incubated with JM1-24-3 for 30 minutes-24 h. Time-dependent reduction in p-AKT (Ser473) and p-mTOR (Ser2448) was observed until 6 hours, while total AKT and total mTOR remained unchanged (FIG. 3C).

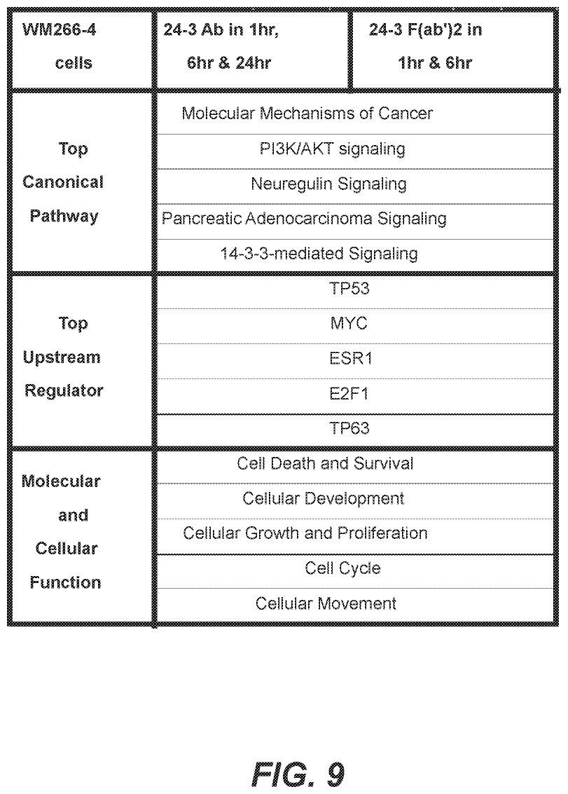

The JM1-24-3 full-length mAb and F(ab′)2 treatment RPPA data together with IPA analysis revealed that JM1-24-3 binding to MUC18 initiated signaling in fundamentally important canonical cancer pathways, including PI3K/AKT and neuregulin; the top associated upstream regulators were TP53, MYC, ESR1 and others; the most important molecular and cellular functions impacted were those associated with cell death and survival, cellular development, and cellular growth and proliferation (FIG. 9).

Through a combination of structural investigations and computational modeling analyses, the inventors defined molecular interactions between JM1-24-3 and its conformational epitope on MUC18. Homology structural modeling results are illustrated in FIG. 3D. The inventors focused on MUC18 conformational changes predicted to result from exposure of the binding epitope to the JM1-24-3 antibody. The inventors assumed that there were multiple confirmations of MUC18 on the cell surface (as for the majority of Ig superfamily adhesion molecules), and that the “bent” form of the MUC18 molecule, which buried the JM1-24-3 epitope, was the preferred, lower energy conformation since it had both less surface solvent exposure as well as more interaction forces. Upon binding of the antibody to the conformational epitope, there was a dynamic transition from the “bent” form to the extended form, which the inventors presumed resulted in downstream signal transduction.

### Example 4—JM1-24-3 Inhibits Melanoma Cell Growth and Blocks Cancer Cell Migration and Invasion by Neutralization of MUC18

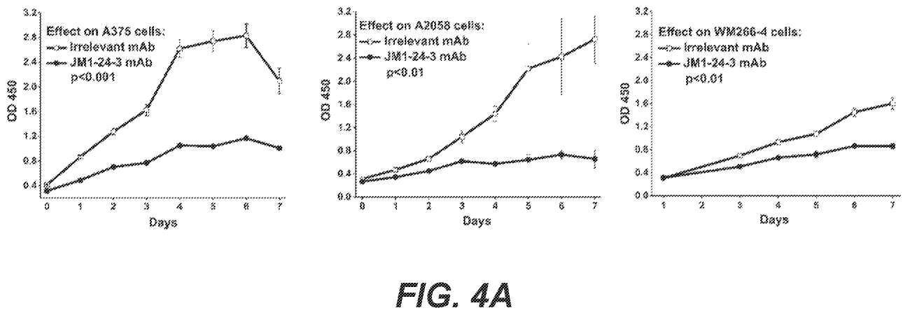

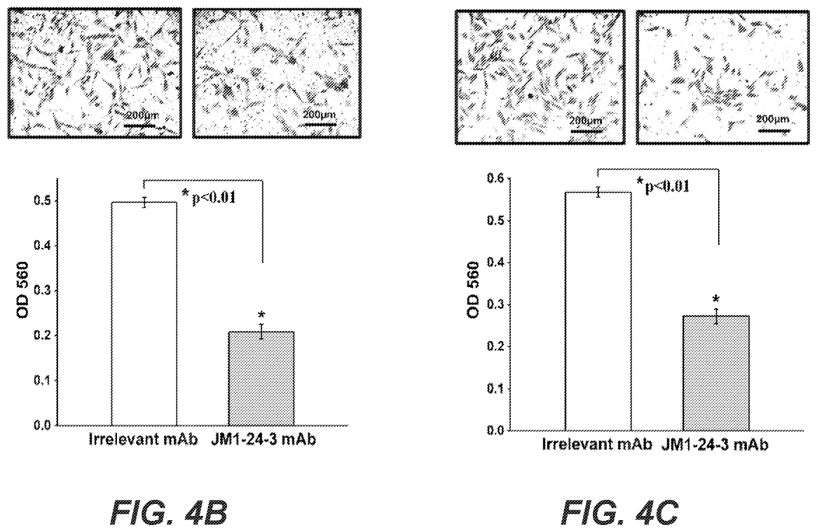

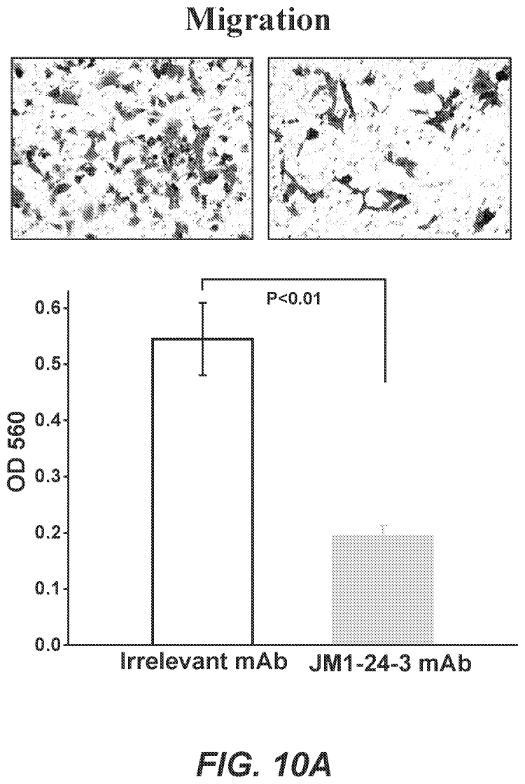

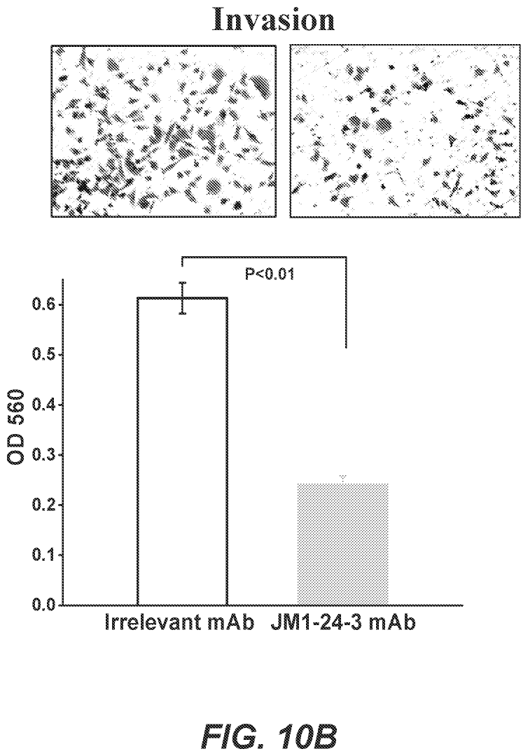

Based on RPPA data, the inventors further performed cell based in vitro assays to evaluate the ability of JM1-24-3 to inhibit melanoma cell growth and proliferation. Proliferation assays showed that JM1-24-3 could significantly inhibit the proliferation of all three melanoma cell lines A375, A2058 and WM266-4 (FIG. 4A) (p<0.01). Migration (FIG. 4B) and invasion (FIG. 4C) assays showed that JM1-24-3 had significant potency and efficacy in inhibition of migration and invasion of WM266-4 cells (p<0.01). Similar effects were observed with A375 cells also (FIGS. 10A-10B).

### Example 5—JM1-24-3 Inhibits Melanoma Tumor Growth and Metastasis in Mouse Xenograft Models

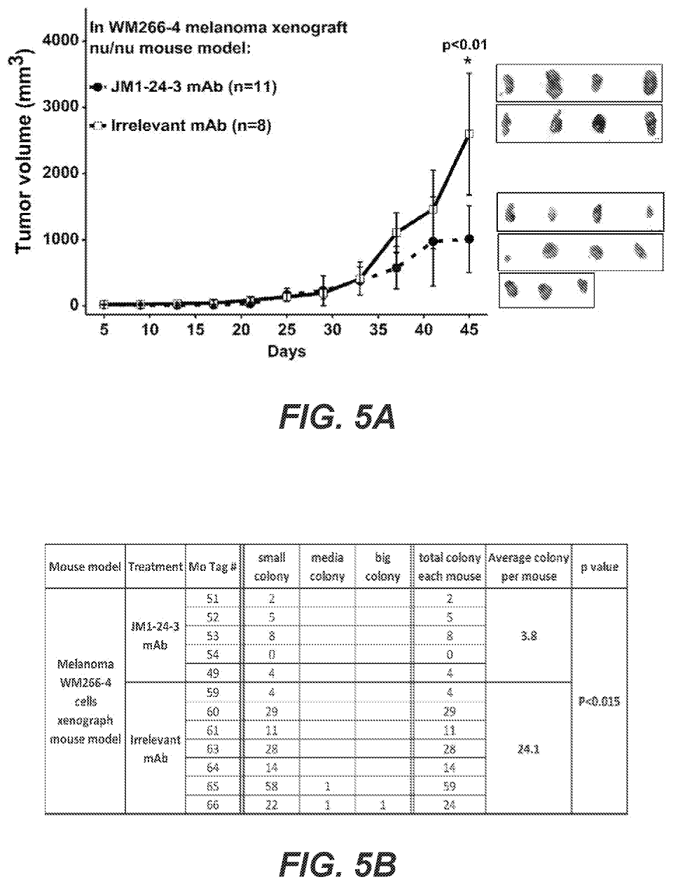

In athymic nude mice, WM266-4 cells (1 million) were injected subcutaneously; after 5 days of inoculation, mice were treated with either JM1-24-3 (n=11) or with irrelevant mAb (n=8) for 45 days (6 mg/kg body weight/i.p./twice per week), and tumor volume was measured with calipers every 4 days. No toxicity was observed, including no reduction in body weight on treatment with JM1-24-3 (data not shown). Tumors were excised and weighed at the end of the experiment. Treatment with JM1-24-3 showed significant reduction in tumor volume (46.8±11.8; p<0.01) as compared to irrelevant mAb treatment (FIG. 5A).

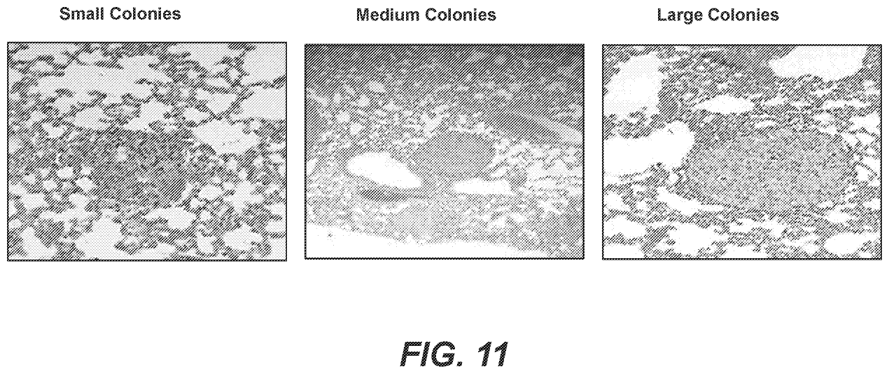

MUC18 expression has been reported to promote hematogenous lung metastasis. The inventors therefore evaluated whether JM1-24-3 could prevent or reduce melanoma lung metastasis in athymic nude mouse xenograft model. Pretreatment with JM1-24-3 (n=5) or irrelevant mAb (n=7) was done one day before tail vein injection of WM266-4 cells (6 mg/kg body weight/i.p./twice per week). All mice were sacrificed at day 45 and their lungs were harvested and stained with H&E (FIG. 11) and the number of metastatic tumor colonies was counted and grouped to small, medium and large according to their sizes. The group treated with JM1-24-3 had significantly fewer colonies than the group treated with irrelevant mAb (p<0.05) (FIG. 5B).

### Example 6—The Expression Levels of MUC18 in Melanoma Have Clinical Significance

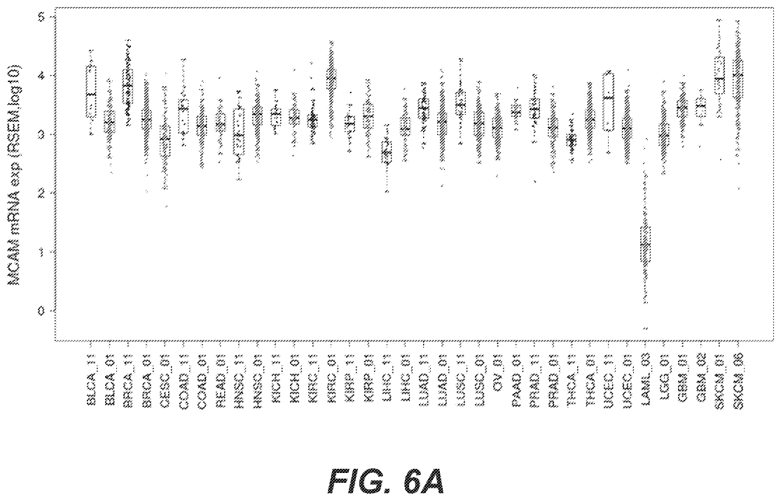

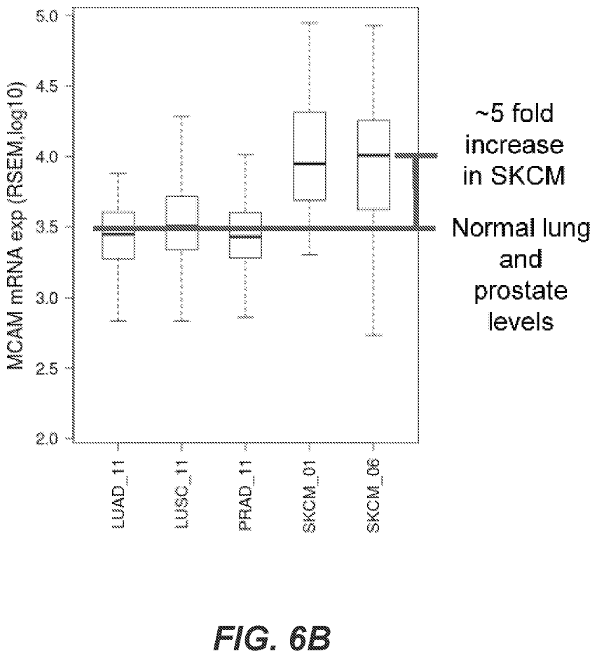

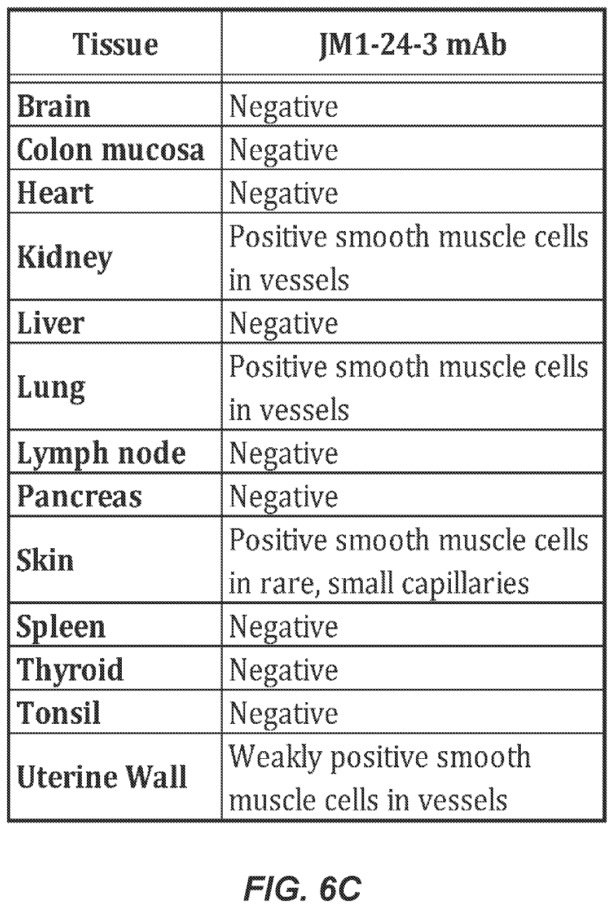

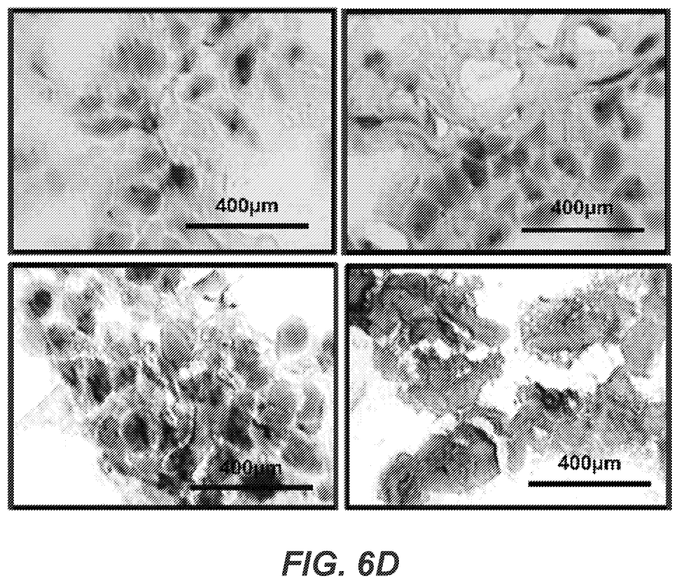

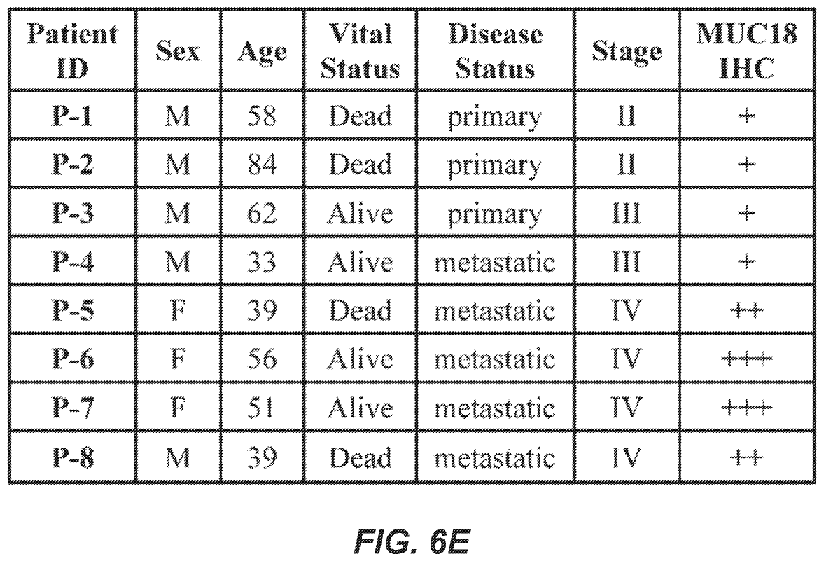

MUC18 mRNA copy numbers were analyzed across a variety of cancers and normal tissues using TCGA dataset and were found to be elevated in both melanoma (SKCM) and renal cell carcinoma (KIRC) (FIG. 6A). Focused comparison using normal lung and prostate tissues versus SKCM illustrated an approximately 5-fold increase of MUC18 mRNA level in SKCM compared to these other tissues (FIG. 6B).

Using TMA IHC, the inventors investigated multiple normal tissues for MUC18 expression as measured by JM1-24-3 binding. Some expression was seen on smooth muscle cells within small vessels in the kidney, lung, skin and uterine wall, but medium and larger sized vessels were uniformly negative, and all other normal tissues evaluated were also negative (FIG. 6C).

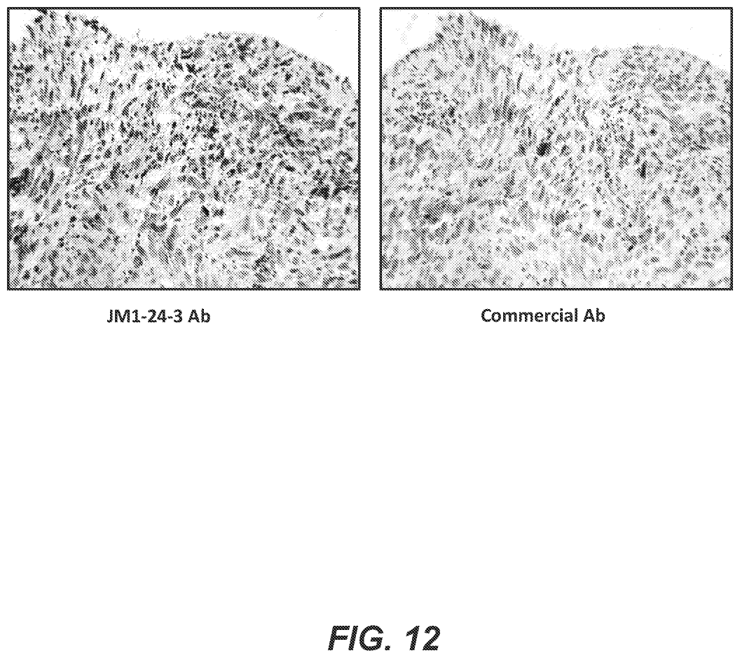

Using JM1-24-3 IHC staining, the inventors investigated tumors from eight melanoma patients with various stages of disease. Representative results of IHC staining are shown in FIG. 6D and the IHC findings were correlated with the clinical status of the patients (FIG. 6E). Although the sample size is small, the results suggest higher expression of MUC18 on melanoma tissues from patients with metastatic melanoma. JM1-24-3 binding was compared with commercially available mouse anti-human MUC18 Ab on IHC on patient tissues. JM1-24-3 showed higher staining intensity and increased staining of cancer cells compared to commercial Ab at the same dilution (FIG. 12).

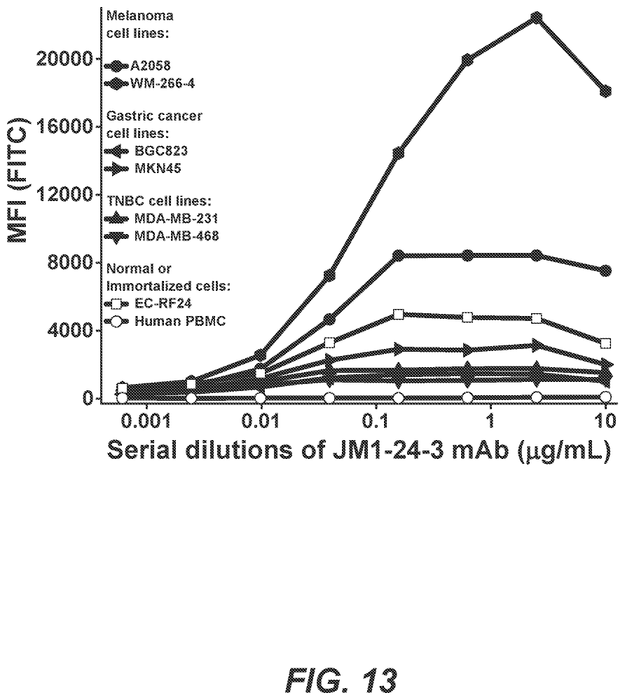

Comparing the binding signals by JM1-24-3 to the surface of various cancer cell lines, melanoma cell lines A2058 and WM266-4 showed the strongest signal, the gastric cancer (GC) cell line MKN45 and EC-RF24 had medium binding signals; triple-negative breast cancer (TNBC) cell lines MDA-MB-231 and MDA-MB-468 and the GC cell line BGC823 had weak binding signals (FIG. 13), whereas human PBMC had no detectable signal.

### Example 7—Exemplary Material and Methods

Animal Study: A/J mice (6-8 weeks old, male) (Harlan Sprague Dawley, Inc., Indianapolis, IN) and nu/nu mice (4-8 weeks old, male) (JAX, Bar Harbor, Maine) were used for this study. Mice were maintained and experiments were performed under protocols (IACUC00001239-RN00 and IACUC00000731-RN01) approved by the Institutional Animal Care and Use Committee (IACUC).

Cell Lines & Tissues & other resources: A375 cells (primary cell line with low metastatic capacity); and A2058 and WM266-4 cells (highly metastatic melanoma cells), SP2/0 (mouse myeloma cells), EC-RF24 (immortalized human endothelial cells) were purchased from American Type Culture Collection (ATCC, Manassas, VA). Human peripheral blood mononuclear cells (PBMCs) were separated by using Ficoll-Plaque Plus (Ficoll, GE Healthcare Biosciences) from healthy donor blood (Gulf Coast Regional Blood Center). Other cell lines were gifts from collaborators. Cell lines were grown in serum free medium MD6 derived from Dulbecco's modified Eagle's medium (DMEM, Gibco) supplemented with 5% FBS and 1% penicillin-streptomycin. Formalin-fixed paraffin embedded (FFPE) tissue slides and tissue microarrays (TMAs) were prepared from a variety of tumors and normal tissues from the inventors' institutional tissue bank under institutional review board protocols (LAB09-0197 and PA11-0957). Tunicamycin (Cat. #T7765, Sigma-Aldrich, MO) and Dynabeads conjugated with Protein A (Cat. #14311D, Invitrogen, Inc., Carlsbad, CA) were also purchased.

Antigen & Antibodies: Human recombinant protein CD146 (MCAM) was purchased from OriGene Technologies (Cat. #TP308937, Rockville, MD). Commercial antibodies used in this study include anti-human MUC18 (mouse mAb, Cat. #MAB932; goat polyclonal Ab, Cat. #AF932, R&D Systems, Inc., Minneapolis, MN; mouse mAb, Cat #ab233923; AbCam, Cambridge, MA) and an irrelevant mAb (served as isotype control antibody) and goat serum (IgG). FITC, HRP and Alexa fluor 647 conjugated goat anti-mouse IgG (Cat. #115-095-071, 115-035-071, 115-606-062, Jackson ImmunoResearch Lab, West Grove, PA) served as secondary antibodies.

Live-cell Immunization: Five million each of A375, A2058 and WM266-4 metastatic melanoma cells were injected subcutaneously (s.c.) into three A/J mice every two weeks×3, followed by an intraperitoneal (i.p.) boost. Three days after boost, spleen cells from the immunized mice were collected and fused with myeloma SP2/0 cells to generate hybridomas.

High-throughput Screening (HTS) Using Fluorescence-activated Cell Sorting (FACS): The BD LSR II Flow Cytometry System with HTS autosampler (Becton Dickinson) was used to screen for mAbs secreted from the hybridomas that bound to the mixed melanoma cell lines as outlined previously. Mean fluorescence intensity (MFI) and the percentage of the stained cell peaks from screening and counter-screening plates were determined using the BD LSR II FACS-HTS system.

Glycosylation and Lectin Binding Analyses: FACS analysis was used to detect the carbohydrate group of the MUC18 glycoprotein from cells (1×105) individually seeded in flasks with or without conditioned medium containing 3.0 μg/mL tunicamycin in DMSO (Dimethyl sulfoxide) and cultured for 24 hours. Cells were then harvested and stained with JM1-24-3 or an irrelevant mAb for quantification of MFI. Cell lysates were probed with JM1-24-3 and 3-actin mAb in WB (western blot). FLISA (Fluorescence-linked immunosorbent assay) was used for SNL (Sambucus nigra lectin) binding analysis, in which individual cell lysates were coated on plates and incubated with SNL-FITC (Fluorescein isothiocyanate) which was then competed by serial dilution of JM1-24-3, starting with 20 μg/mL, with or without SNL. Similar FLISA assays were conducted for other lectins [DSA (Datura stramonium agglutinin) lectin and DSA-FITC; LCA (Lens culinaris agglutinin) lectin and LCA-FITC; WGA (Wheat germ agglutinin) lectin and WGA-FITC].

Epitope Mapping: The biochip was spotted in triplicate with MUC18 8-mer and 6-mer oligopeptides at one amino acid resolution (acetyl capped at the N-termini on the ChipMDA_130046) and then incubated with JM1-24-3 (1.0 μg/mL) at 4° C. for 2 hours, followed by washing and incubation with goat anti-mouse IgGFc Alexa 647 conjugated (0.01 μg/mL) at 4° C. for 2 hours, and then washed again. The image of the biochip was scanned in Cy5 channel and data analyzed.

Immunohistochemistry (IHC) Analysis: The expression of MUC18 on melanoma patient tissues and on TMA containing several normal tissues and cancers was detected by IHC using JM1-24-3 mAb (1:1,000) as described previously.

Immunoprecipitation (IP) and Mass Spectrometry (MS): IP was conducted on cell lysates (100 μg) using Protein A beads (0.2 mL) and JM1-24-3 (10 μg) or irrelevant control antibodies as described previously. Excised bands were analyzed with a ProteinChip system in Series 4000 Mass Spectrometry (Bio-Rad) as described previously.

Reverse Phase Protein Array (RPPA): WM266-4 cells were treated with JM1-24-3 or its F(ab′)2 fragment for 1 hr or 6 hrs and RPPA conducted. Heat map results were analyzed for changes in protein expression using Ingenuity Pathway Analysis (IPA) software (Qiagen Bioinformatics) to identify down-stream signaling pathways.

Structural Modeling: The homology model of the extracellular domain (residues 5-559) of MUC18 was generated using the Swiss Model web interface, using the functional motif of “search for templates”. The first approach modes were used to generate an initial homology model and accompanying sequence alignment, which was then manually modified using Coot. A final correction of the alignment was then performed in Swiss Pdb Viewer. This model and the accompanying corrected sequence alignment were then input into the Optimization Mode of Swiss Model. The final pdb files were used to display the overall conformation and the antibody epitope locations.

The homology model of JM1-24-3 variable domain was generated using BioLuminate v.1.1 (Schrödinger, New York, NY). Template coordinates for heavy chain and light chain were chosen from PDB structure 4BKL and 6I1O respectively based on the sequence homology search. CDRs were modeled using the BioLuminate Basic Loop Model function. Experimental data were incorporated into the program for interaction. All antibody structure images were generated using Maestro v.9.4 and PyMol (Schrödinger, New York, NY).

In vitro Cell Proliferation, Migration and Invasion Studies: These studies were conducted on incubating cells with and without JM1-24-3 or irrelevant mAb (150 μg/mL) for one week as described previously. For migration assays, 3×104 cells in serum-free medium (300 μL) were incubated with and without JM1-24-3 (150 μg/mL) or irrelevant mAb for 24 hours. Invasion assays were carried out by a similar protocol using QCM ECMatrix cell Invasion Assay Kit (EMD Millipore).

In vivo Tumor Xenograft Studies of Melanoma Growth and Metastasis: Athymic nude (nu/nu) mice were injected subcutaneously with WM266-4 cells (1 million/0.1 mL). After 5 days mice were randomly divided into two groups and treated with either JM1-24-3 (6 mg/kg body weight/i.p./twice a week) (n=11) or with the same dose of irrelevant mAb (n=8) for 45 days. Tumor volume was measured with calipers every 4 days. At the end of the experiment, tumors were excised and weighed. Mice body weight was measured every 4 days and any reduction >10% from initial weight was considered as toxicity. For metastasis studies, mice were pre-treated one day before with JM1-24-3 (n=5) or with irrelevant mAb (n=7), following which all received a tail vein injection with WM266-4 cells (1 million); mAb treatment was administered every 4 days. All mice were sacrificed at day 45 and lungs were harvested for H&E staining.

Statistical analysis: Experiments were repeated at least in replicate and data expressed as mean±standard deviation (SD). Representative figures are presented for FACS, ELISA, FLISA and MS analysis. Differences were analyzed with a two-tailed Student's t-test or Wilcoxon rank-sum test and paired t-test for serial dilution studies. P values<0.05 were considered as statistically significant.

All of the methods disclosed and claimed herein can be made and executed without undue experimentation in light of the present disclosure. While the compositions and methods of this disclosure have been described in terms of certain embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

The references recited in the application, to the extent that they provide exemplary procedural or other details supplementary to those set forth herein, are specifically incorporated herein by reference.

What is claimed is:

1. A polypeptide that specifically binds to MUC18, comprising:
(a) a heavy chain variable region (V_{H}) comprising:
(i) a CDR-H1 comprising a sequence having at least 85% identity to SEQ ID NO:1, SEQ ID NO:2, or SEQ ID NO:3;
(ii) a CDR-H2 comprising a sequence having at least 85% identity to SEQ ID NO:4, SEQ ID NO:5, or SEQ ID NO:6; and
(iii) a CDR-H3 comprising a sequence having at least 85% identity to SEQ ID NO:7, SEQ ID NO:8, SEQ ID NO:9, or SEQ ID NO:10; and
(b) a light chain variable region (V_{L}) comprising:
(i) a CDR-L1 comprising a sequence having at least 85% identity to SEQ ID NO:11, SEQ ID NO:12, or SEQ ID NO:13;
(ii) a CDR-L2 comprising a sequence having at least 85% identity to SEQ ID NO:14, SEQ ID NO:15, or SEQ ID NO:16 and
(iii) a CDR-L3 comprising a sequence having at least 85% identity SEQ ID NO:17, SEQ ID NO:18, or SEQ ID NO:19.

2. The polypeptide of claim 1, wherein the CDR-H1 comprises SEQ ID NO:1.

3. The polypeptide of claim 1, wherein the CDR-H1 comprises SEQ ID NO:2.

4. The polypeptide of claim 1, wherein the CDR-H1 comprises SEQ ID NO:3.

5. The polypeptide of any one of claims 1-4, wherein the CDR-H2 comprises SEQ ID NO:4.

6. The polypeptide of any one of claims 1-4, wherein the CDR-H2 comprises SEQ ID NO:5.

7. The polypeptide of any one of claims 1-4, wherein the CDR-H2 comprises SEQ ID NO:6.

8. The polypeptide of any one of claims 1-7, wherein the CDR-H3 comprises SEQ ID NO:7.

9. The polypeptide of any one of claims 1-7, wherein the CDR-H3 comprises SEQ ID NO:8.

10. The polypeptide of any one of claims 1-7, wherein the CDR-H3 comprises SEQ ID NO:9.

11. The polypeptide of any one of claims 1-7, wherein the CDR-H3 comprises SEQ ID NO:10.

12. The polypeptide of any one of claims 1-11, wherein the CDR-L1 comprises SEQ ID NO:11.

13. The polypeptide of any one of claims 1-11, wherein the CDR-L1 comprises SEQ ID NO:12.

14. The polypeptide of any one of claims 1-11, wherein the CDR-L1 comprises SEQ ID NO:13.

15. The polypeptide of any one of claims 1-14, wherein the CDR-L2 comprises SEQ ID NO:14.

16. The polypeptide of any one of claims 1-14, wherein the CDR-L2 comprises SEQ ID NO:15.

17. The polypeptide of any one of claims 1-14, wherein the CDR-L2 comprises SEQ ID NO:16.

18. The polypeptide of any one of claims 1-17, wherein the CDR-L3 comprises SEQ ID NO:17.

19. The polypeptide of any one of claims 1-17, wherein the CDR-L3 comprises SEQ ID NO:18.

20. The polypeptide of any one of claims 1-17, wherein the CDR-L3 comprises SEQ ID NO:19.

21. The polypeptide of any one of claims 1-20, wherein the V_{H} comprises a sequence having at least 85% identity to SEQ ID NO:20.

22. The polypeptide of any one of claims 1-21, wherein the V_{H} comprises a sequence having at least 95% identity to SEQ ID NO:20.

23. The polypeptide of any one of claims 1-22, wherein the V_{H} comprises SEQ ID NO:20.

24. The polypeptide of any one of claims 1-23, wherein the V_{L} Comprises a sequence having at least 85% identity to SEQ ID NO:21.

25. The polypeptide of any one of claims 1-24, wherein the V_{L} Comprises a sequence having at least 95% identity to SEQ ID NO:21.

26. The polypeptide of any one of claims 1-25, wherein the V_{L} Comprises SEQ ID NO:21.

27. The polypeptide of any one of claims 1-26, wherein the polypeptide specifically binds to an extracellular domain of MUC18.

28. The polypeptide of any one of claims 1-27, wherein the polypeptide preferentially binds to a glycosylated MUC18 compared to a non-glycosylated MUC18.

29. The polypeptide of any one of claims 1-28, wherein the polypeptide has an association constant for a MUC18 protein of between about 1 and 3 nM.

30. The polypeptide of any one of claims 1-29, wherein the polypeptide is an antibody or antigen-binding fragment thereof.

31. The polypeptide of any one of claims 1-30, wherein the polypeptide is a human antibody, humanized antibody, recombinant antibody, chimeric antibody, an antibody derivative, a veneered antibody, a diabody, a monoclonal antibody, or a polyclonal antibody.

32. The polypeptide of claim 30 or claim 31, wherein the polypeptide is a monoclonal antibody.

33. The polypeptide of any one of claims 30-32, wherein the polypeptide is a murine antibody.

34. The polypeptide of any one of claims 30-33, wherein the polypeptide is JM1-24-3.

35. The polypeptide of claim 30 or claim 31, wherein the polypeptide is a humanized antibody.

36. A nucleic acid encoding for the polypeptide of any one of claims 1-35.

37. The nucleic acid of claim 36, wherein the nucleic acid comprises a sequence having at least 85% identity to SEQ ID NO:22.

38. The nucleic acid of claim 36 or claim 37, wherein the nucleic acid comprises a sequence having at least 95% identity to SEQ ID NO:22.

39. The nucleic acid of any one of claims 36-38, wherein the nucleic acid comprises SEQ ID NO:22.

40. The nucleic acid of claim 36, wherein the nucleic acid comprises a sequence having at least 85% identity to SEQ ID NO:23.

41. The nucleic acid of claim 36 or claim 40, wherein the nucleic acid comprises a sequence having at least 95% identity to SEQ ID NO:23.

42. The nucleic acid of any one of claim 36 or claims 40-41, wherein the nucleic acid comprises SEQ ID NO:23.

43. A vector comprising the nucleic acid of any one of claims 36-42.

44. A cell comprising the polypeptide of any one of claims 1-35, the nucleic acid of any one of claims 36-42, or the vector of claim 43

45. The cell of claim 44, wherein the cell is an immune cell.

46. The cell of claim 44 or claim 45, wherein the cell is a T cell.

47. The cell of claim 44 or claim 45, wherein the cell is a B cell.

48. A method for generating a polypeptide, the method comprising culturing the cell of any one of claims 44-47 under conditions sufficient to express the nucleic acid in the cell.

49. A method for generating the polypeptide of any one of claims 1-35, the method comprising (a) providing a nucleic acid encoding for the polypeptide to a cell, and (b) subjecting the cell to conditions sufficient to express the polypeptide from the nucleic acid.

50. A pharmaceutical composition comprising:
(a) the polypeptide of any one of claims 1-35, the nucleic acid of any one of claims 36-42, the cell of any one of claims 44-47, or the vector of claim 43; and
(b) a pharmaceutically acceptable excipient.

51. The pharmaceutical composition of claim 50, further comprising an additional therapeutic.

52. The pharmaceutical composition of claim 51, wherein the additional therapeutic is a chemotherapeutic.

53. A method for treating a subject for cancer, the method comprising administering to the subject a therapeutically effective amount of a composition comprising the polypeptide of any one of claims 1-35, the nucleic acid of any one of claims 36-42, the cell of any one of claims 44-47, or the vector of claim 43.

54. The method of claim 53, wherein the cancer is a MUC18+ cancer.

55. The method of claim 53 or 54, wherein the cancer is melanoma.

56. The method of any one of claims 53-55, further comprising administering to the subject an additional therapy.

57. The method of claim 56, wherein the additional therapy is radiotherapy, chemotherapy, or immunotherapy.

58. An anti-MUC18 antibody or antigen-binding fragment thereof, comprising:
(a) a V_{H} comprising:
(i) a CDR-H1 comprising SEQ ID NO:1;
(ii) a CDR-H2 comprising SEQ ID NO:4; and
(iii) a CDR-H3 comprising SEQ ID NO:7; and
(b) a V_{L} comprising:
(i) a CDR-L1 comprising SEQ ID NO:11;
(ii) a CDR-L2 comprising SEQ ID NO:14; and
(iii) a CDR-L3 comprising SEQ ID NO:17.

59. The anti-MUC18 antibody or antigen-binding fragment thereof of claim 58, wherein the V_{H} comprises SEQ ID NO:20.

60. The anti-MUC18 antibody or antigen-binding fragment thereof of claim 58 or claim 59, wherein the V_{L} comprises SEQ ID NO:21.

61. The anti-MUC18 antibody or antigen-binding fragment thereof of any one of claims 58-60, wherein the anti-MUC18 antibody or antigen-binding fragment thereof has an association constant for a MUC18 protein of between about 1 and 3 nM.


SEQUENCE LISTING (24 sequences)

SEQ ID NO: 1 - protein, 8 aa, Artificial Sequence
  Synthetic Polypeptide
GYAFTSYW

SEQ ID NO: 2 - protein, 10 aa, Artificial Sequence
  Synthetic Polypeptide
GYAFTSYWMN

SEQ ID NO: 3 - protein, 9 aa, Artificial Sequence
  Synthetic Polypeptide
YAFTSYWMN

SEQ ID NO: 4 - protein, 8 aa, Artificial Sequence
  Synthetic Polypeptide
IDPFNGYT

SEQ ID NO: 5 - protein, 10 aa, Artificial Sequence
  Synthetic Polypeptide
AIDPFNGYTE

SEQ ID NO: 6 - protein, 14 aa, Artificial Sequence
  Synthetic Polypeptide
WIGAIDPFNGYTEY

SEQ ID NO: 7 - protein, 2 aa, Artificial Sequence
  Synthetic Polypeptide
AR

SEQ ID NO: 8 - protein, 9 aa, Artificial Sequence
  Synthetic Polypeptide
WGGRLYFDY

SEQ ID NO: 9 - protein, 10 aa, Artificial Sequence
  Synthetic Polypeptide
RWGGRLYFDY

SEQ ID NO: 10 - protein, 11 aa, Artificial Sequence
  Synthetic Polypeptide
ARWGGRLYFDY

SEQ ID NO: 11 - protein, 6 aa, Artificial Sequence
  Synthetic Polypeptide
QDINSF

SEQ ID NO: 12 - protein, 11 aa, Artificial Sequence
  Synthetic Polypeptide
KASQDINSFLS

SEQ ID NO: 13 - protein, 8 aa, Artificial Sequence
  Synthetic Polypeptide
QDINSFLS

SEQ ID NO: 14 - protein, 3 aa, Artificial Sequence
  Synthetic Polypeptide
RAN

SEQ ID NO: 15 - protein, 7 aa, Artificial Sequence
  Synthetic Polypeptide
RANRLVD

SEQ ID NO: 16 - protein, 11 aa, Artificial Sequence
  Synthetic Polypeptide
TLIYRANRLVD

SEQ ID NO: 17 - protein, 7 aa, Artificial Sequence
  Synthetic Polypeptide
LQYDEFP

SEQ ID NO: 18 - protein, 9 aa, Artificial Sequence
  Synthetic Polypeptide
LQYDEFPYT

SEQ ID NO: 19 - protein, 8 aa, Artificial Sequence
  Synthetic Polypeptide
LQYDEFPY

SEQ ID NO: 20 - protein, 108 aa, Artificial Sequence
  Synthetic Polypeptide
MSCKASGYAFTSYWMNWIKERPGQGLEWIGAIDPFNGYTEYNHKFKDKAILTADNSSSTVYMQLSSLTSEDSAVYYCARWGGRLYFDYWGQGTTLTVSSAKTTPPSDY

SEQ ID NO: 21 - protein, 110 aa, Artificial Sequence
  Synthetic Polypeptide
MTQSPSSMYASLGERVTITCKASQDINSFLSWFQQKPGKSPKTLIYRANRLVDGVPSRFSGSGSGQDYSLTISSLEYEDMGIYYCLQYDEFPYTFGGGTKLEIKRADAAP

SEQ ID NO: 22 - DNA, 255 bp, Artificial Sequence
  Synthetic Polynucleotide
atgtcctgcaaggcttctggctacgcctttactagttactggatgaactggataaaagagaggcctggacagggtctggaatggattggggctattgatcctttcaatggttatactgagtacaatcataagttcaaggacaaggccatattgactgcagacaattcctccagcacagtctacatgcaactgagcagcctgacatctgaggactctgcagtctattactgtgcaagatgggggggacgtctctac

SEQ ID NO: 23 - DNA, 255 bp, Artificial Sequence
  Synthetic Polynucleotide
atgacccagtctccatcttccatgtatgcatctctaggagagagagtcactatcacttgcaaggcgagtcaggacattaatagttttttaagctggttccagcagaaaccagggaaatctcctaagaccctgatctatcgtgcaaacagattggtagatggggtcccatcaaggttcagtggcagtggatctgggcaagattattctctgaccatcagcagcctggagtatgaagatatgggaatttattactgt

SEQ ID NO: 24 - protein, 646 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKSELVVEVKSDKLPEEMGLLQGSSGDKRAPGDQGEKYIDLRH